(19) United States
(12) Patent Application Publication
(10) Pub. No.: US 2007/0055352 A1
(43) Pub. Date: Mar. 8, 2007

(54) Stent with pockets for containing a therapeutic agent

(76) Inventors: Wendy Naimark, Cambridge, MA (US); Peter Shank, Boylston, MA (US); Maria Palasis, Wellesley, MA (US); Toby Freyman, Waltham, MA (US); Anastasia Panos, Athens (GR); Samuel J. Epstein, Watertown, MA (US); Alexandra Rousseau, Cambridge, MA (US)

Correspondence Address: JONES DAY, 222 EAST 41ST ST, NEW YORK, NY, 10017, US

(21) Appl. No.: 11/221,647

(22) Filed: Sep. 7, 2005

(51) Int. Cl. A61F 2/90

(52) U.S. Cl. 623/1.16; 623/1.42; 623/1.44

(57) ABSTRACT

A medical device for: delivering a therapeutic agent to a body lumen is described. The device may generally be a stent with an inner sidewall surface and an outer sidewall surface, with a plurality of struts having a plurality of openings therein. An inner and outer layer may be applied to the stent, forming pockets within at least a portion of the openings. The pockets may be filled with at least one therapeutic agent. The pockets may take a variety of shapes and sizes, and may be designed for release or rupture in variety of ways.

### FIELD OF THE INVENTION

This invention relates generally to medical devices, such as stents, for delivering a therapeutic agent to body tissue of a patient, such as a body lumen. More particularly, the invention is directed to a stent comprising at least one pocket for containing a therapeutic agent as well as ways for making such stents. The invention is also directed to a method for delivering therapeutic agents to body tissue of a patient.

### BACKGROUND OF THE INVENTION

A variety of medical conditions have been treated by introducing an insertable medical device having a coating for release of a therapeutic agent. For example, various types of medical devices coated with a therapeutic agent, such as stents, have been proposed for localized delivery of such agents to a body lumen. See, e.g., U.S. Pat. No. 6,099,562 to Ding et al. issued on Aug. 8, 2000. However, it has been noted that therapeutic agent delivery by means of medical devices can be improved.

In particular, the effectiveness of coated medical devices is limited by the surface area of the medical device. This problem is exacerbated when the medical device is used to delivery biopharmaceuticals, such as gene therapies and proteins. Generally, biopharmaceuticals have large therapeutic application windows. The use of coated medical devices makes the upper areas of these windows difficult or impossible to explore and test because of the limited carrying capacity of a coated medical device. The present invention provides a medical device that has increased carrying capacity to address this and other needs.

### SUMMARY OF THE INVENTION

The present invention seeks to address these needs by providing a stent having struts with pockets between at least one strut having at least one therapeutic agent.

In one embodiment, a medical device is provided for delivering a therapeutic agent comprising: (a) a stent having a sidewall comprising a plurality of struts, at least a first opening in the sidewall, and a first sidewall surface at least partially defined by the plurality of struts and the first opening; (b) a first layer disposed over at least a part of the first sidewall surface, wherein at least a portion of the first layer extends over a part of the first opening; (c) a second layer disposed over at least a part of the first sidewall surface, wherein at least a portion of the second layer is disposed over the portion of the first layer that extends over the first opening, (d) at least a first pocket disposed about at least a portion of the first opening; wherein the pocket is defined at least in part by the first layer and at least in part by the second layer; and (e) a therapeutic agent contained in the first pocket.

In another embodiment, a medical device is provided for delivering a therapeutic agent comprising: (a) a stent having a sidewall comprising a plurality of struts, at least a first opening in the sidewall, an outer sidewall surface at least partially defined by the plurality of struts and the first opening, and an inner sidewall surface at least partially defined by the plurality of struts and the first opening; (b) a first layer disposed over at least a portion of the outer sidewall surface, wherein at least a portion of the first layer extends over a part of the first opening, and wherein the first layer is bound to at least a portion of the stent; (c) a second layer disposed over at least a portion of the inner sidewall surface, wherein at least a portion of the second layer extends over the first opening, (d) at least a first pocket disposed about at least a portion of the opening; wherein the pocket is defined at least in part by the first layer and at least in part by the second layer; and (e) a therapeutic agent contained in the first pocket.

In another embodiment, a medical device is provided for delivering a therapeutic agent comprising: (a) a stent having a sidewall comprising a plurality of struts, at least a first opening in the sidewall, an outer sidewall surface defined by the plurality of struts and the opening, and an inner sidewall surface defined at least partially by the plurality of struts and the opening; (b) a first layer disposed over at least a portion of the outer sidewall surface, wherein at least a portion of the first layer extends over a part of the first opening; (c) a second layer disposed over at least a part of the inner sidewall surface, wherein at least a portion of the second layer extends over the opening, and wherein the second layer is bound to at least a portion of the first layer, (d) at least a first pocket disposed about at least a portion of the opening; wherein the pocket is defined at least in part by the first layer and at least in part by the second layer; and (e) a therapeutic agent contained in the first pocket.

In yet another embodiment, A medical device for delivering a therapeutic agent comprising: (a) a stent comprising a sidewall comprising at least a first strut and a second strut, and at least a first opening in the sidewall, wherein the first strut and the second strut each comprise an outer surface, an inner surface and at least one side surface; (b) a first layer bound to the side surface of at least one of the first and second struts, wherein at least a portion of the first layer extends over a portion of the first opening; (c) a second layer bound to the side surface of at least one of the first and second struts, wherein at least a portion of the second layer extends over a portion of the first opening; (d) at least a first pocket disposed about at least a portion of the opening; wherein the pocket is defined at least in part by the first layer and at least in part by the second layer; and (e) a therapeutic agent contained in the first pocket. The medical device of claim 1, wherein the first layer is bound to the stent.

The second layer may be bound to the first layer. The second layer may be disposed over at least part of the first layer that is disposed over the first sidewall surface.

The first pocket may be co-extensive with the opening. The first pocket may be within the first opening. The first pocket may extend beyond the first opening. The medical device may further comprise a second pocket disposed about the opening.

At least one strut may comprise a side surface. The first and/or second layer may be disposed over the side surface.

The sidewall may further comprise a second sidewall surface. The first and/or second layer may be disposed over the second sidewall surface.

The stent further may comprise a second opening. A second pocket may be disposed about the second opening. The second pocket may contain a therapeutic material. The second pocket may contain a different therapeutic material than the first pocket. The second pocket may be disposed about the first opening. The first and second pockets may be interconnected.

At least one of the first and second layers may comprise a plurality of sub-layers. At least two sub-layers may be comprised of a different material. At least two sub-layers may be of different thicknesses.

The medical device may further comprise a barrier between the first and second layers. The medical device may further comprise a third layer.

The first and second layers may be comprised of the same material, or different materials. The first and second layers may have different tensile strengths. The first and second layers may be of different thicknesses.

At least one of the first and second layer may be capable of being ruptured by the expansion of the stent. At least a portion of at least one of the first and second layer may comprise a plurality of pores. At least one of the first layer and second layer may comprise at least one preformed imprint. The imprinted area may generally have a lower tensile strength than the remainder of the layer.

At least one of the first layer and second layer may comprise a self-sealing material. At least one of the first layer and second layer may comprise a biodegradable material. At least one of the first layer and second layer may be substantially flexible.

The therapeutic agent may be releasable from the first pocket through at least one of the first layer and second layer. The therapeutic agent may be releasable from the first pocket after the expansion of the stent.

A method for making a medical device is also provided comprising the steps of: (a) providing a stent comprising a sidewall having an inner surface, an outer surface, at least one opening in the sidewall; wherein the sidewall comprises a plurality of struts, wherein the struts have an outer surface, an inner surface, and at least one side surface; (b) applying a first layer to a surface of the sidewall, and bonding at least a portion of the first layer to a surface of at least one strut, and covering at least one opening; (c) applying a second layer to a surface of the sidewall and bonding at least a portion of the second layer to a surface of at least one strut, and covering at least one opening, forming at least one pocket is generally disposed in at least one opening.

Another method for making a medical device comprising the steps of: (a) providing a prefabricated stent having an inner surface, an outer surface, and a sidewall comprising a plurality of struts having a plurality of openings therein; (b) applying a first layer disposed on the inner surface to form a covering over least a portion of the inner surface and at least one of the openings therein, so that at least a portion of the first layer is bonded to at least a portion of the inner surface; (c) applying a second layer disposed on the outer surface, so that at least a portion of the second layer is bonded to at least a portion of the outer surface, and so that at least one opening is located between the first layer and the second layer to form at least one pocket between the struts.

Another method of making a medical device is described comprising the steps of: (a) providing a stent comprising a sidewall having a first surface, a second surface, at least one opening in the sidewall; wherein the sidewall comprises a plurality of struts, wherein the struts have at least one surface; (b) applying a first layer about the first surface of the sidewall, and covering at least one opening; (c) applying a second layer to at least a portion of the first layer, and covering at least one opening, forming at least one pocket is generally disposed about at least one opening.

Another method of making a medical device is described comprising the steps of: (a) providing a stent comprising a sidewall having a first surface, a second surface, at least one opening in the sidewall; wherein the sidewall comprises a plurality of struts, wherein the struts have at least one surface; (b) applying a first layer to the first surface of the sidewall, and bonding at least a portion of the first layer to a surface of at least one strut, and covering at least one opening; (c) applying a second layer to the second surface of the sidewall, bonding at least a portion of the second layer to the surface of at least one strut, and covering at least one opening, forming at least one pocket is generally disposed about at least one opening.

Another method of making a medical device is described comprising the steps of: (a) providing a stent comprising a sidewall having a first surface, a second surface, at least one opening in the sidewall; wherein the sidewall comprises a plurality of struts, wherein the struts have at least one surface; (b) applying a first and second layers about the first surface of the sidewall, covering at least one opening, and forming at least one pocket is generally disposed about at least one opening.

Another method of making a medical device is described comprising the steps of: (a) providing a stent comprising a sidewall having a first surface, a second surface, at least one opening in the sidewall; wherein the sidewall comprises a plurality of struts, wherein the struts have at least one side surface; (b) applying a first and second layers about at least one side surface of at least one strut, covering at least one opening, and forming at least one pocket is generally disposed about at least one opening.

The method may further comprise the step of applying at least one therapeutic agent to at least a portion of the stent. The method may further comprise the step of inserting at least one therapeutic agent into at least one pocket.

The method may further comprise forming at least one imprint on the first and/or second layer. At least one imprint may be formed using a mandrel.

The method may further comprise the step of coating at least one of the first layer and second layer with a therapeutic agent.

### BRIEF DESCRIPTION OF THE DRAWINGS

Preferred features of the present invention are disclosed in the accompanying drawings, wherein similar reference characters denote similar elements throughout the several views, and wherein:

FIG. 1A is a side view of an exemplary stent suitable for the present invention;

FIG. 1B is a partial cross-sectional view of the stent of FIG. 1A along line A-A;

FIG. 2A is a partial side view of an exemplary stent where pockets are disposed within the openings;

FIG. 2B is a partial side view of an exemplary stent where pockets extend to the boundaries of the openings;

FIG. 2C is a partial side view of an exemplary stent where two or more pockets are disposed about an opening;

FIG. 2D is a partial side view of an exemplary stent where a pocket is disposed about more than one opening;

FIGS. 3A-3B are partial cross-sectional views of an exemplary stent with pockets formed by layers disposed along the outer sidewall of the stent;

FIGS. 3C-3D are partial cross-sectional views of an exemplary stent with pockets formed by layers disposed along the inner sidewall of the stent;

FIG. 4 is a partial cross-sectional view of an exemplary stent with pockets formed both by layers disposed along the outer sidewall of the stent and the inner sidewall of the stent;

FIGS. 5-7 are a partial cross-sectional view of an exemplary stent with further embodiments of pockets formed by layers disposed along a sidewall of the stent;

FIGS. 8A-8O are enlarged partial cross-sectional views of pockets formed from two layers disposed in openings between adjacent struts;

FIGS. 9A-9C are enlarged partial cross-sectional views of pockets formed from three layers disposed in openings between adjacent struts;

FIGS. 10A-10F are partial cross-sectional views of an exemplary stent with pockets formed by layers disposed on both the outer and inner sidewalls of the stent;

FIGS. 11A-11R are enlarged partial cross-sectional views of pockets formed from at least two layers disposed in openings between adjacent struts;

FIG. 12A is an exemplary cross-sectional view of a stent with struts in a compressed state;

FIG. 12B shows the stent of FIG. 12A with a first layer;

FIG. 12C shows the stent of FIG. 12B with amounts of therapeutic material placed at or near the first layer;

FIG. 12D shows the stent of FIG. 12C with a second layer, forming pockets around the amounts of therapeutic material;

FIG. 12E shows the stent of FIG. 12D in an expanded state, the second layer having ruptures;

FIG. 12F shows the stent of FIG. 12E with the content of the pockets dispersing;

FIGS. 13A-13F is another embodiment of the method described in FIGS. 12A-12F; and

FIGS. 14A-14D are enlarged partial cross-sectional views of pockets formed from two layers disposed in openings between adjacent struts, wherein the adjacent struts are of varying shapes and sizes.

### DETAILED DESCRIPTION OF THE INVENTION

A. Suitable Stents

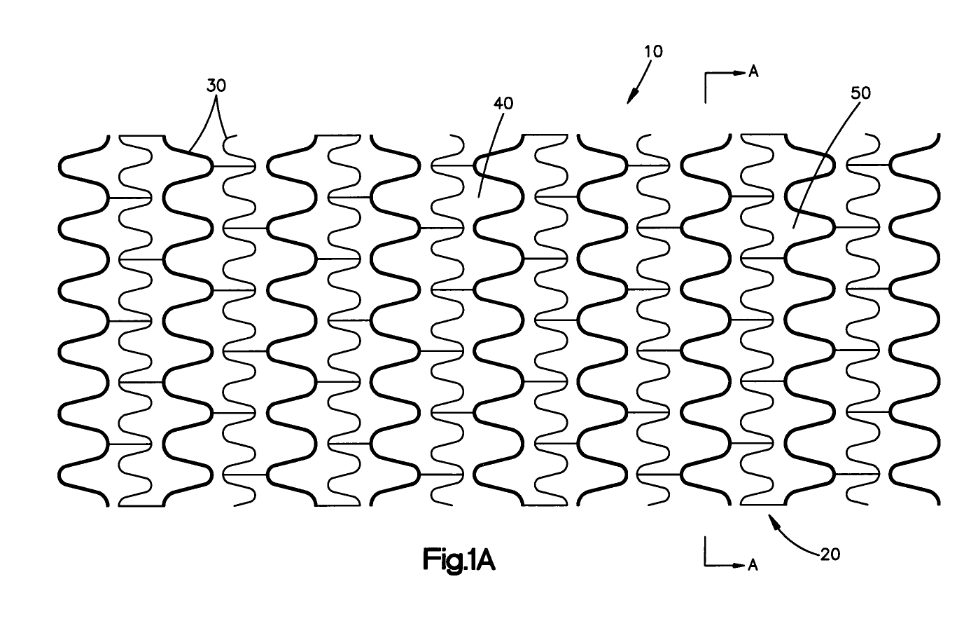

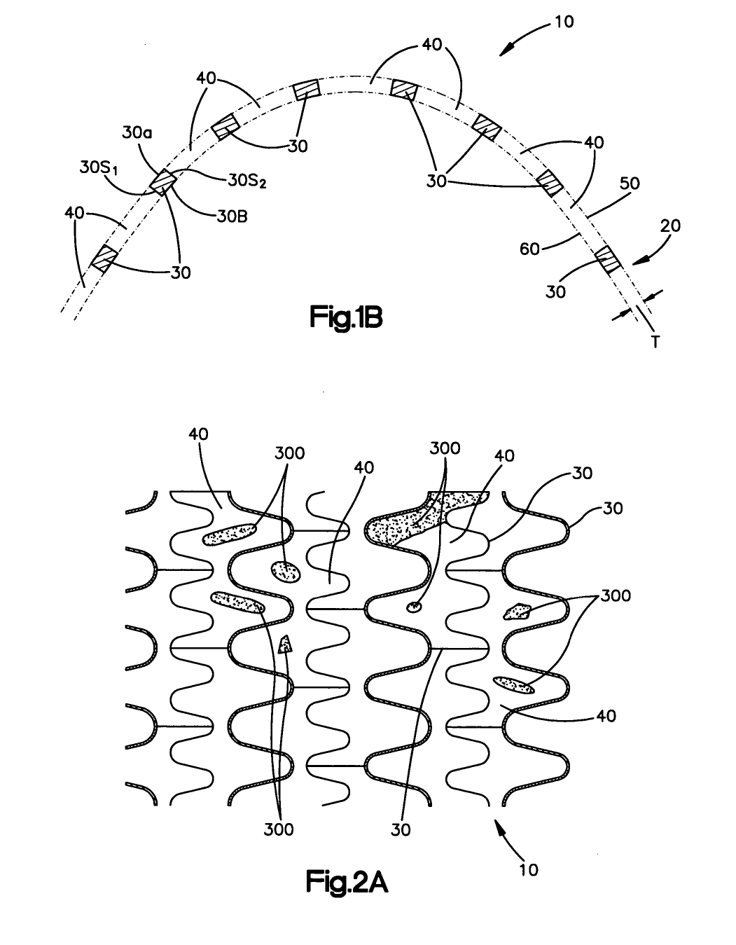

The invention described in detail herein generally relates to a stent having at least one opening in which at least one pocket is disposed about the opening. Suitable stents include ones that are used for cardiovascular, urinary and other medical applications. FIG. 1A shows an example of a stent suitable for the present invention. In this example, the stent 10 comprises a sidewall 20 which comprises a plurality of struts 30 and at least one opening 40 in the sidewall 20. Generally, the opening 40 is disposed between adjacent struts 30. Also, the sidewall 20 may have a first sidewall surface 50 and an opposing second sidewall surface 60, which is not shown in FIG. 1A. The first sidewall surface 50 can be an outer sidewall surface, which faces the body lumen wall when the stent is implanted, or an inner sidewall surface, which faces away from the body lumen wall. Likewise, the second sidewall surface 60 can be an outer sidewall surface or an inner sidewall surface. If the first sidewall surface is the outer sidewall surface, the second sidewall surface is the inner sidewall surface. If the first sidewall surface is the inner sidewall surface, the second sidewall surface is the outer sidewall surface.

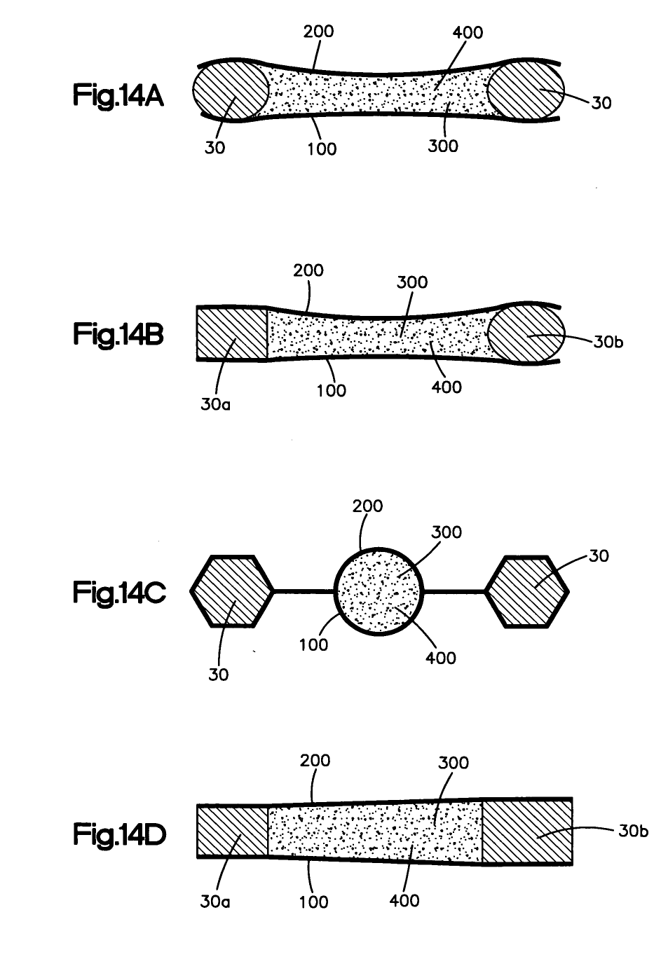

FIG. 1B shows a cross-sectional view of the stent 10 in FIG. 1A along line A-A. The sidewall 20 may comprise a plurality of struts 30. Each strut 30 may have an outer surface 30u, which may generally be the surface of the strut 30 that faces the body lumen wall when the stent is implanted, and an inner surface 30i, which may generally be the surface facing away from the body lumen wall when the stent is implanted. Struts 30 may also have side surfaces 30s₁, 30s₂, which may be disposed between the outer and inner strut surfaces 30u, 30i. The cross-sections of the struts 30 can be of any suitable shape (see, infra, FIGS. 14A-14D).

As shown in FIG. 1A, the sidewall 20 has a thickness T. Furthermore, the sidewall may have a first sidewall surface 50, which in this example is the outer surface of the sidewall. The first sidewall surface 50 may be defined by the openings 40 and the struts 30. The sidewall may also have a second sidewall surface 60, which in this example is the inner surface of the sidewall. The strut outer surface 30u may generally lie along the outer sidewall surface 50. The strut inner surface 30i may generally lie along the inner sidewall surface 60.

Other suitable stents include, for example, intravascular stents such as those described in U.S. Pat. No. 6,478,816 to Kveen et al., for “Stent”, issued on Nov. 12, 2002, incorporated herein by reference in its entirety. Suitable stents include self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

Stents that are suitable for the present invention may be fabricated from metallic, ceramic, or polymeric materials, or a combination thereof. Metallic materials are more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Suitable ceramic materials include, but are not limited to, oxides, carbides, or nitrides of the transition elements such as titaniumoxides, hafnium oxides, iridiumoxides, chromium oxides, aluminum oxides, and zirconiumoxides. Silicon based materials, such as silica, may also be used.

The polymer(s) useful for forming the stent should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for stents include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

B. The Pockets

Pockets 300 may be disposed in openings 40. As discussed in greater detail below, pockets 300 may be of various shapes and sizes. Pockets 300 may also be situated about openings 40 in a variety of manners. A single stent 10 may have several different types of pockets 300. Numerous variations and applications will be appreciated by those skilled in the art.

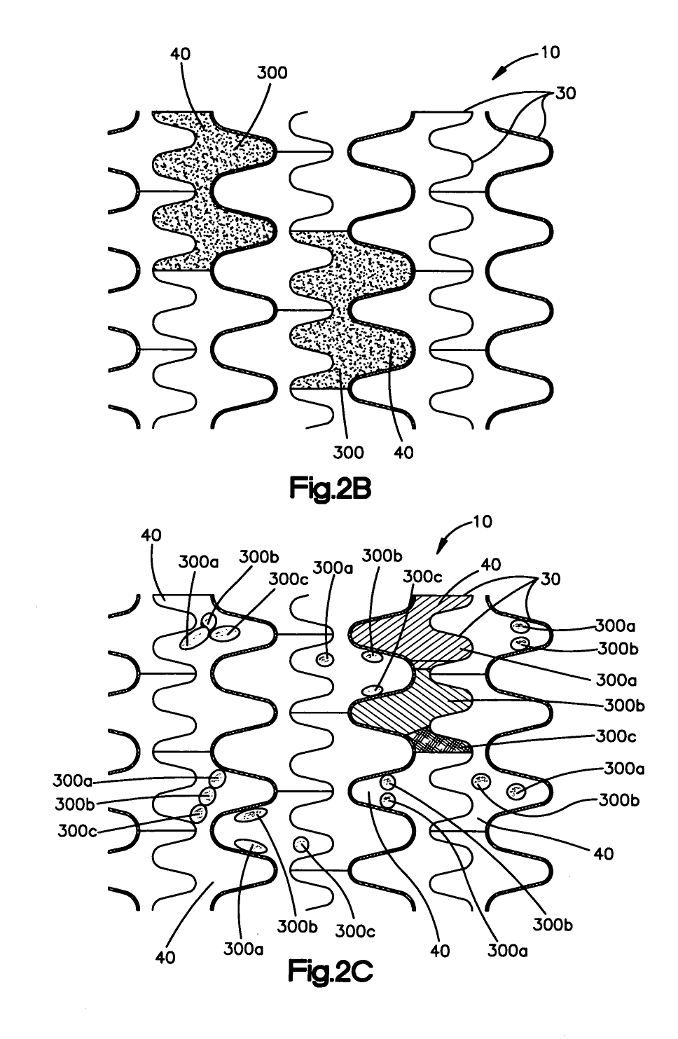

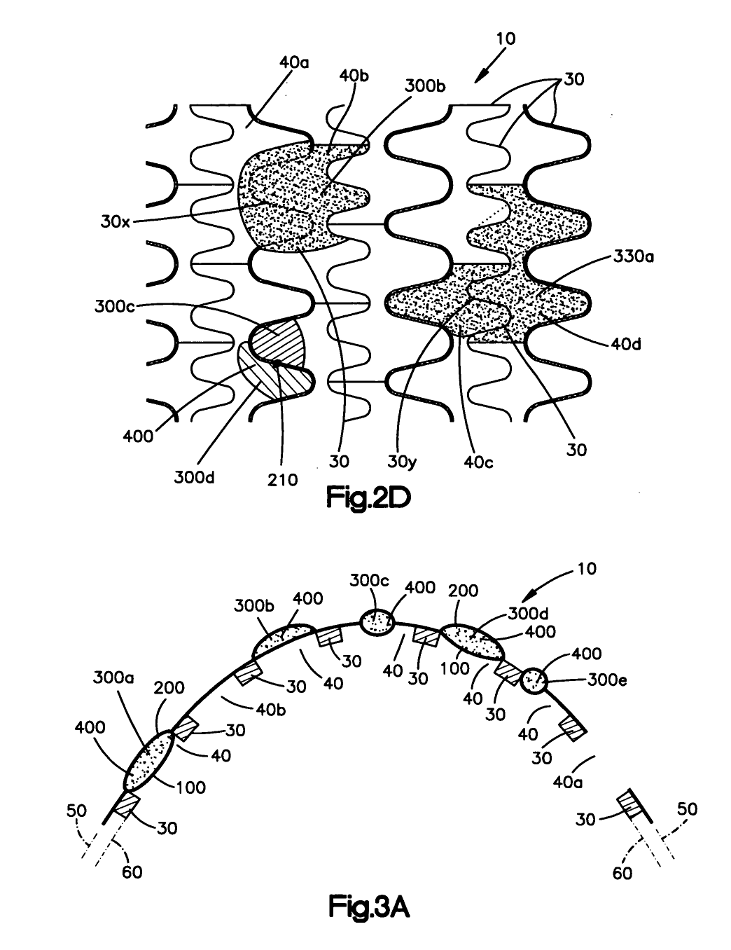

FIG. 2A shows exemplary pockets 300 disposed about the openings 40 in the present invention can be situated completely within the boundaries of the openings. These pockets 300 do not contact the boundaries of the openings 40, which are generally defined by struts 30. In other embodiments, such as those shown in FIG. 2B, the pockets 300 can contact boundaries of the openings 40, which are generally defined by the struts 30. In some such embodiments, the pockets 300 may be coextensive with the opening 40.

In yet other embodiments, such as those shown in FIG. 2C, a plurality of pockets 300a, 300b, 300c can be disposed about a single opening 40. The number of pockets 300 that can be disposed about an opening 40 can vary from opening to opening within a stent 10. For example, a first pocket 300a can be disposed about a first opening 40 in a stent 10, while, two or more pockets 300b, 300c can be disposed about a second opening 40. When two or more pockets 300 are disposed about an opening 40, the pockets can have various shapes and sizes as shown in FIG. 2C. Also, the two or more pockets 300 disposed about an opening 40 can be separated from each other or, some or all of the pockets can be in contact with each other.

In other embodiments, a pocket 300 can be disposed about two or more openings 40a, 40b as shown in FIG. 2D. Pockets 300 can be coextensive with some or all of the openings 40, as shown with pocket 300a in FIG. 2D. Alternatively, the pocket 300 can be disposed about only portions of the openings 40, such as shown in pocket 300b. Also, in some embodiments, the pocket 30 can be disposed over the outer sidewall surface, as shown in pocket 300b so that strut 30x is covered by the pocket 300b. In other embodiments, the pocket 300a can be disposed over the inner sidewall surface, so that strut 30y lies above the pocket 300a. In yet other embodiments, the pocket 300 can be disposed about a strut 30 so that a part of the pocket is disposed over the strut and part of the pocket is disposed under the pocket (discussed in more detail below).

As also shown in FIG. 2D, combined amounts of therapeutic material 400 disposed within pockets 300c, 300d may be in communication with each other by way of an orifice 210. At least one therapeutic agent may be present in an amount of therapeutic material 400. The orifice 210 may be open or closed during the implantation of the stent 10. The amounts of therapeutic material comprising a combined amount of therapeutic material 400 are in at least two different openings 40. Combined amounts of therapeutic material 400 may be beneficial to combine the contents of more than one amount of therapeutic material together upon release of the contents in the body. For instance, it may be beneficial for two amounts of therapeutic material 400a, 400b comprising a combined amount of therapeutic material 400 to be initially provided with two separate substances, but desirable for those substances to remain at least partially separate until the substances are released from the individual amounts of therapeutic material. For example, first and second adjacent amounts of therapeutic material 400a, 400b may comprise a combined amounts of therapeutic material 400, wherein the first adjacent amount of therapeutic material 400a is comprised with a inactive cells and the second adjacent pocket is filled with active genes. Upon expansion of the stent 10 (discussed in detail below), the orifice 210 may rupture allowing the contents of the first and second adjacent amounts of therapeutic material 400a, 400b combine before the combined amount of therapeutic material 400 itself ruptures and releases its combined contents into the body.

C. Pockets With Layers Disposed Over the Same Sidewall Surface

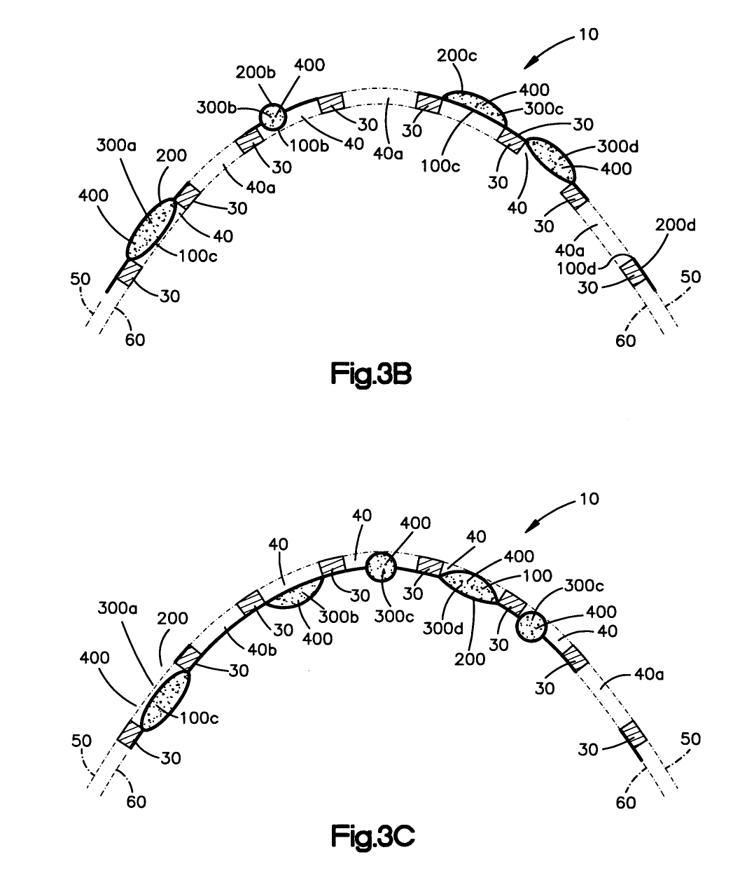

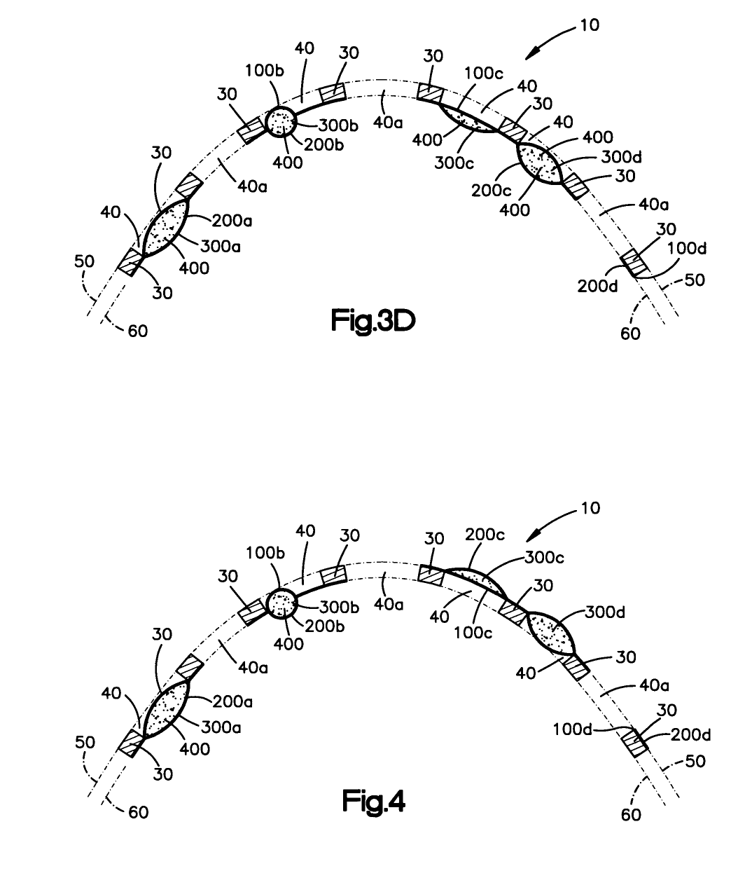

In one embodiment of the invention, a first layer 100 is disposed over at least a part of a first sidewall surface, which can be the outer or inner sidewall surface 50, 60. At least a portion of the first layer 100 may extend over a part of an opening 40. Moreover, a second layer 200 may also be disposed over at least a part of the first sidewall surface. At least a portion of the second layer 200 may be disposed over the portion of the first layer 100 that extends over the opening 40. The first layer 100 may define a first surface of a pocket 300 and the second layer may define a second surface of the pocket 300. The pocket 300 may be disposed about at least a portion of the opening 40. A therapeutic agent 400 can be contained in the pocket 300. FIGS. 3A-3D shows an example of such an embodiment where the layers defining the pocket are disposed over the same stent sidewall surface.

FIG. 3A shows a cross-section of a stent 10 with pockets 300. In this example the first layer 100 and second layer 200 define a number of pockets 300a, 300b, 300c, 300d and 300e. The pockets may contain a therapeutic agent 400. In this embodiment, both the first layer 100 and the second layer 200 are disposed over the same sidewall surface, which in this example is the outer sidewall surface 50. Even though the first and second layers 100, 200 are disposed over the outer sidewall surface 50, the pockets defined by the first and second layers 100, 200 can extend into the openings 40, such as pockets 300a, 300c, 300d and 300e.

As discussed above, the pockets 300 can contact the boundaries of the openings 40 about which they are disposed. Pockets 300a and 300d in FIG. 3A are examples of such pockets. Pockets 300c is an example of a pocket that is disposed within a single opening 40 and does not contact the boundaries of the opening 40. Although the pockets 300 may be disposed within an opening 40, the pocket can extend above or below the opening, such as the tops of the pockets 300, which may be defined by the first layer 100. In addition, pockets 300 can be disposed about an opening 40 when the pocket is disposed over or under an opening such as pocket 300b. Also, in a stent with pockets 300, some openings 40 can be free of pockets, such as opening 40a. Furthermore, although the first and second layers 100, 200 can extend over an opening 40, the layers 100, 200 do not have to form a pocket, such as in opening 40b.

FIG. 3B shows embodiments of pockets 300 similar to those shown in FIG. 3A, but with some openings 40a free of pockets spaced between pockets 300a-300e. The result of this arrangement is that separate sets of first 100a, 100b, 100c and second layers 200a, 200b, 200c may define one or more pockets 300. In this embodiment, first and second layers 100a, 200a define pocket 300a, first and second layers 100b, 200b, which can be referred to as third and fourth layers, define pocket 300b, and first and second layers 100c, 200c, which can be referred to as fifth and sixth layers, define pockets 300c, 300d.

FIG. 3C shows a cross-section of a stent 10 with pockets 300 similar to those shown in FIG. 3A. However, in this embodiment, the first and second layers 100, 200 are disposed over the inner sidewall surface 60. FIG. 3D again shows embodiments of pockets 300 similar to those shown in FIG. 3C, but with more openings 40a free of pockets spaced between pockets 300a-300d. As previously discussed, first and second layers 100, 200 may form numerous pockets 300.

FIG. 4 shows another embodiment of a stent 10 with pockets 300. In this embodiment, different sets of first and second layers 100, 200 are disposed over the inner sidewall surface 60 at one or more locations to form pockets 300a, 300b, and also disposed over the outer sidewall surface 50 at one or more locations to form pockets 300c, 300d. In this embodiment, first and second layers 100a, 100b form pocket 300a, which is coextensive over an opening 40 on inner sidewall surface 60. Layers 100b, 200b form a bulbous pocket 300b, which is off-center in opening 40 on inner sidewall surface 60. Layers 100c, 200c collectively form two pockets 300c, 300d disposed along outer sidewall surface 50.

FIG. 5 shows another embodiment of the inventions. In this embodiment, neither the first and second layers 100, 200 are entirely disposed over a common opening 40. For instance, first layer 100a is disposed over opening 40v and the two adjacent struts 30. However, second layer 200a is disposed over first layer 100a, but not over the two adjacent struts 30 that are adjacent opening 40v. Likewise, pocket 300b is defined by second layer 200b, which is disposed over two adjacent struts 30 and first layer 100b, which is not disposed over the two adjacent struts 30 adjacent opening 40w. Similarly, pockets 300c and 300d are formed by a first or second layer that is disposed over just one strut that is adjacent the respective opening over which each pocket is disposed. Lastly, pocket 300e is defined by a first layer 100d, which is disposed over one strut 30 adjacent opening 40z, and a second layer 200d, which is disposed over two adjacent struts 30 adjacent opening 40z over which pocket 300e is disposed.

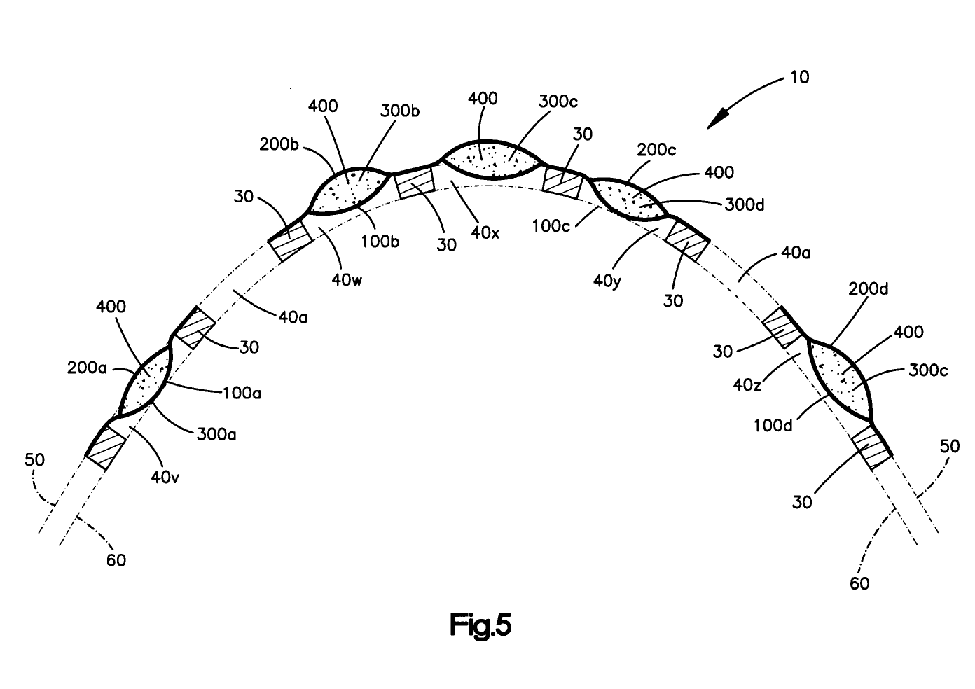

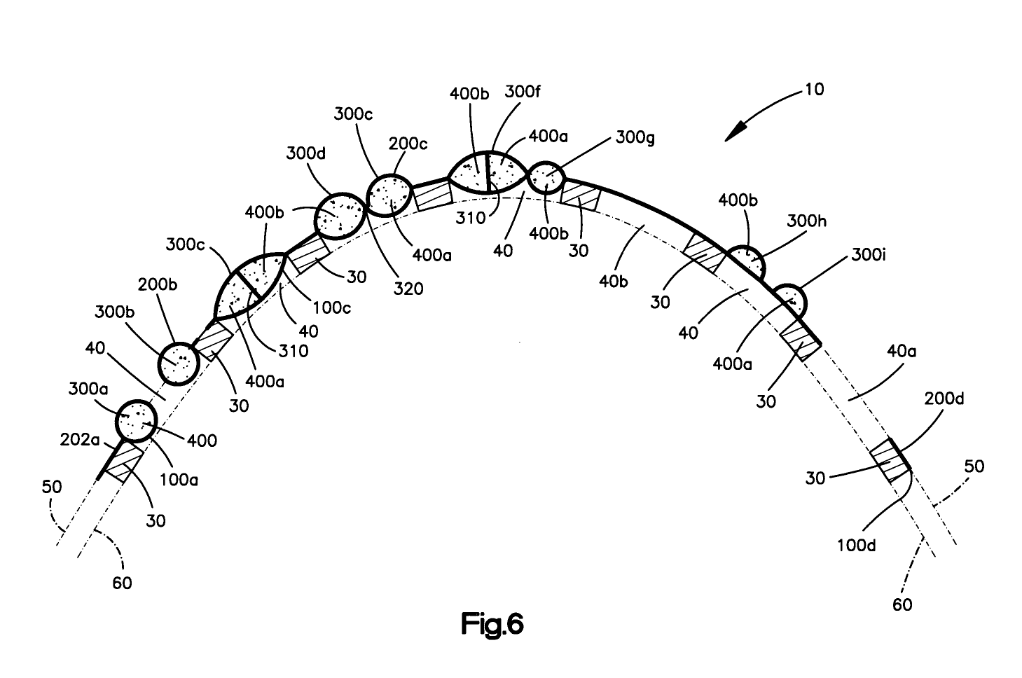

More than one pocket 300 may be situated about a single opening 40. As seen in FIG. 6, pockets 300a, 300b are both situated about opening 40. Moreover, pockets 300a, 300b may not be in contact with each other, thereby each being formed by separate first 100a, 100b and second layers 200a, 200b.

FIG. 6 also shows an embodiment of a stent 10 with pockets 300 wherein at least one pocket has more than one therapeutic material 400a, 400b contained within it. An exemplary pocket 300 with more than one therapeutic material is seen in pocket 300c. Therapeutic materials 400a, 400b may be separated by a barrier 310 situated within a pocket 300. Barrier 310 may be rupturable.

FIG. 6 further shows embodiments of therapeutic materials 400a, 400b contained in pairs of separate pockets 300d, 300e and 300h, 300i. Each pair of pockets may be situated about a single opening 40. Combinations of the above-described pockets are also contemplated for a single opening, as seen with pockets 300f and 300g.

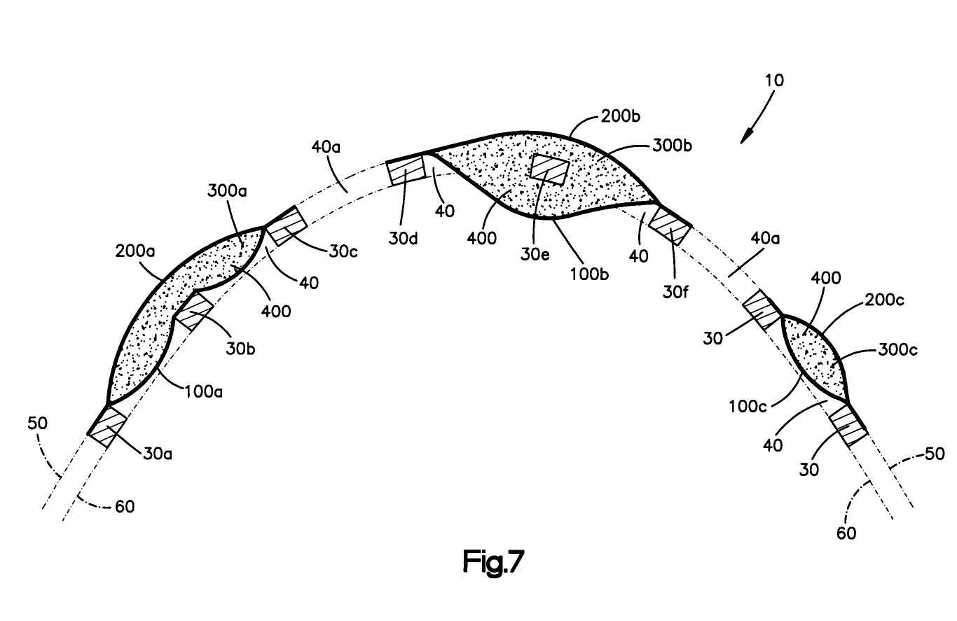

FIG. 7 shows another embodiment of a stent 10 with pockets 300. In this embodiment, pockets 300a, 300b are situated about more than one opening 40. Pocket 300a is formed by first layer 100a, which is in contact with three consecutive struts 30a, 30b, 30c and second layer 200a, which contacts first layer 100a only at or near struts 30a and 30c. Pocket 300b is formed in a similar manner across struts 30d, 30e, 30f, except that in this embodiment, intermediate strut 30e is enclosed within pocket 300b, as first layer 100b passes below strut 30e, and second layer 200b passes above strut 30e. Pockets 300a, 300b may therefore be larger than a pocket 300 formed solely in a single opening 40.

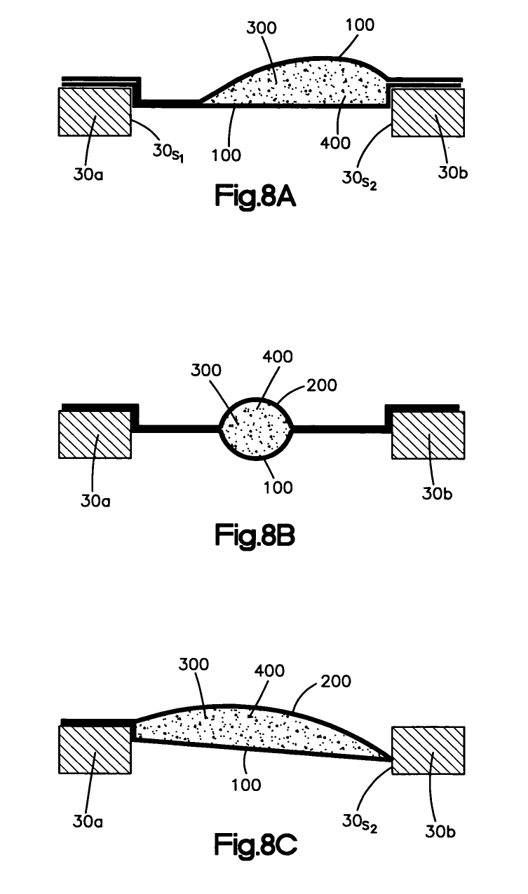

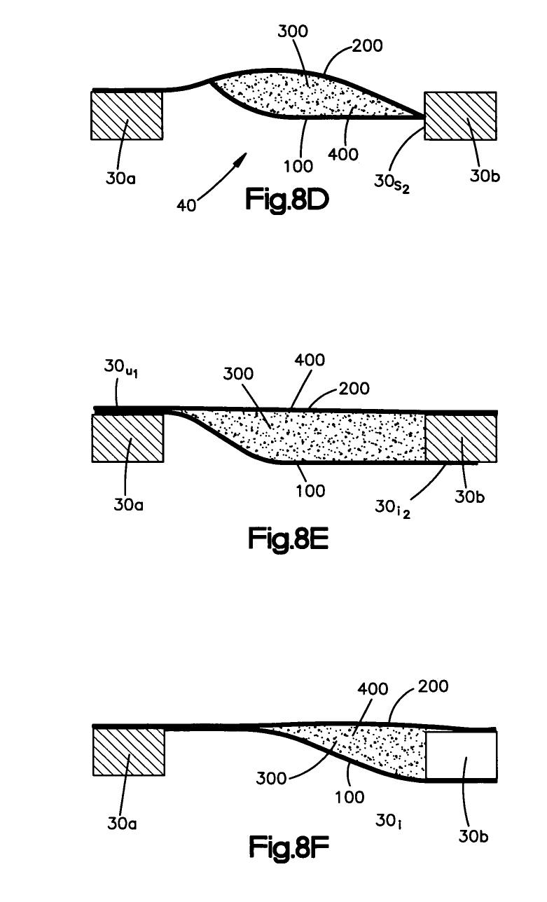

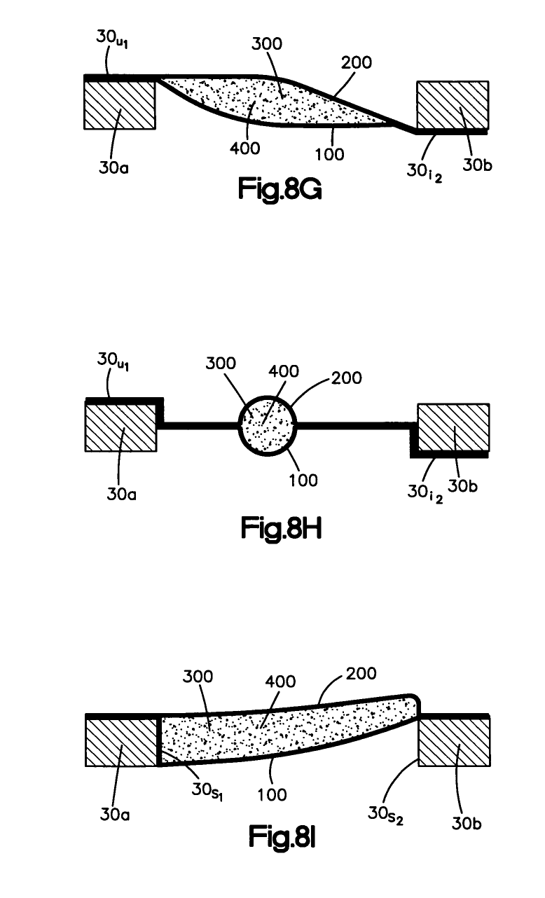

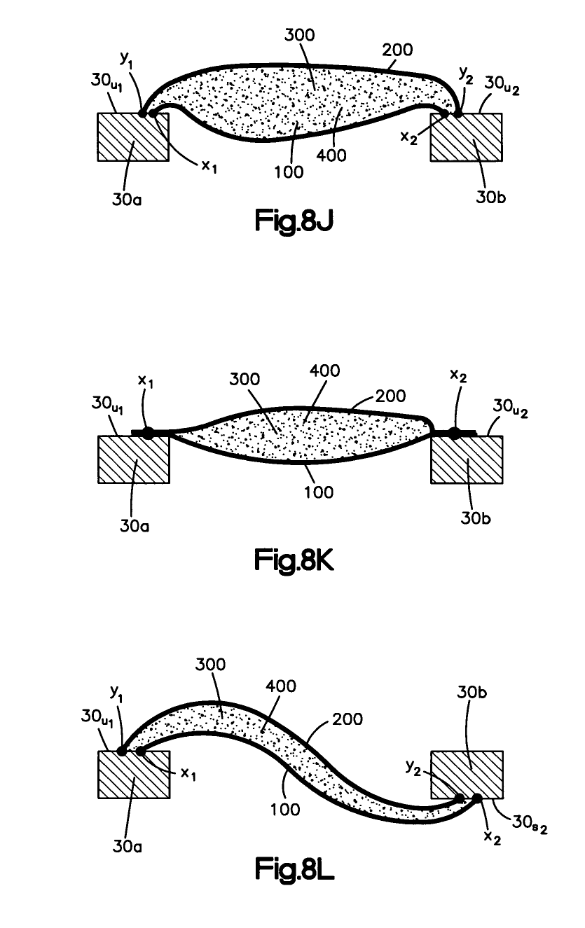

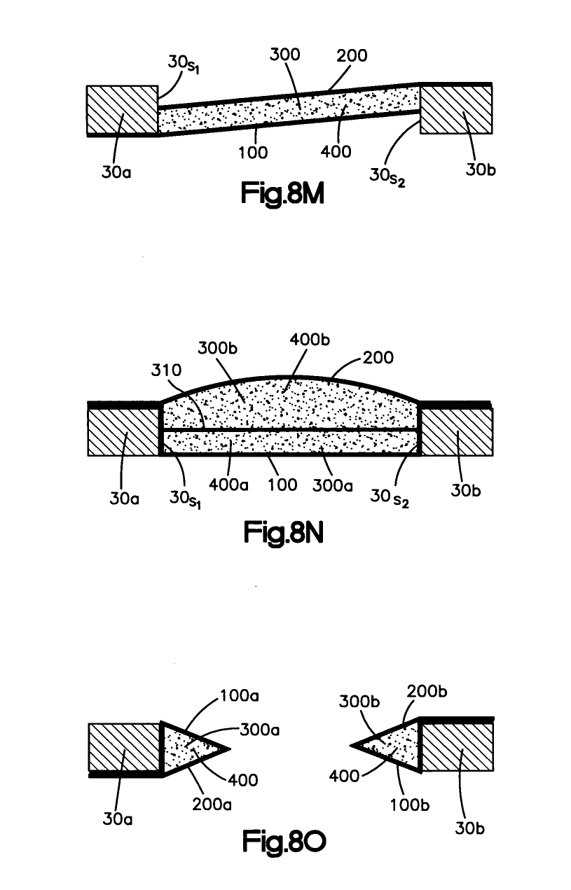

FIGS. 8A-8O are enlarged partial cross-sectional views of pockets 300 formed from first and second layers 100, 200 disposed in openings 40 between adjacent struts 30a, 30b. It is expressly contemplated that a pocket 300 could exhibit some or all of the characteristics of pockets described herein, and in detail below.

FIG. 8A shows a pocket 30 formed by first and second layers 100, 200 partially conforming to side surfaces 30s₁ and 30s₂ of adjacent struts 30a, 30b, wherein the second layer 200 has a rise and first layer 100 is generally flat.

FIG. 8B shows a pocket 300 formed by first and second layers 100, 200, wherein the pocket is generally formed by a circular bulge spaced apart from adjacent struts 30a, 30b.

FIG. 8C shows a pocket 300 formed by first and second layers 100, 200, wherein a boundary of the pocket is formed by layers 100, 200 terminating at side surface 30s₂ of strut 30b.

FIG. 8D shows a pocket 300 formed by first and second layers 100, 200, wherein first and second layers 100, 200 terminate at side surface 30s₂ of strut 30b, and first layer 100 does not extend across the entire opening 40 between adjacent struts 30a, 30b.

FIG. 8E shows a pocket 300 formed by first and second layers 100, 200, wherein first and second layers 100, 200 are situated on the outer surface 30u, of adjacent strut 30a, but the first layer 100 is in contact with the inner surface 30i₂ of adjacent strut 30b, while second layer 200 is in contact with the outer surface 30u₂ of adjacent strut 30b. The arrangement seen about strut 30b is discussed in more detail, infra. FIG. 8F shows a pocket 300 formed by a combination of the embodiments shown in FIGS. 8D and 8E.

FIG. 8G shows a pocket 300 formed by first and second layers 100, 200 that are situated at or near outer surface 30u, of adjacent strut 30a, and then at or near inner surface 30i₂ of adjacent strut 30b. FIG. 8H shows a pocket formed by a combination of the embodiments shown in FIGS. 8B and 8G.

FIG. 8I shows a pocket 300 formed by first and second layers 100, 200, wherein the first layer 100 is in contact with the entire side surface 30s₁ of adjacent strut 30a.

FIGS. 8J-8L show embodiments of pockets 300 formed by first and second layers 100, 200, wherein the first and second layers are bonded to surfaces of adjacent struts 30a, 30b and/or each other. Layers 100, 200 in FIG. 8J are individually bonded to the outer surfaces 30u₁, 30u₂ of adjacent struts 30a, 30b, respectively, First layer 100 may be bonded to outer surfaces 30u₁, 30u₂ at points x₁, x₂, respectively. Second layer 200 may be bonded to outer surfaces 30u₁, 30u₂ at points y₁, y₂, respectively. Layers 100, 200 in FIG. 8K are bonded to each other instead of adjacent struts 30a, 30b, at points x₁ and x₂.

FIG. 8L combines the embodiments of FIGS. 8G and 8J. Layers 100, 200 may be bonded to the outer surface 30u₁ of strut 30a at points x₁, y₁, respectively, but then bonded to the inner surface 30i₂ of strut 30b at points x₂, y₂, respectively. The resulting arrangement is a “wave” pattern.

FIG. 8M shows a pocket 300 formed by first and second layers 100, 200 wherein the first layer 100 contacts the inner surface 30i, and is bonded to the side surface 30s₂ of strut 30b, and the second layer 200 is bonded to the side surface 30s₁ of strut 30a and contacts the outer surface 30u₂ of strut 30b. The resultant arrangement is a “slanted” pattern.

FIG. 8N shows pockets 300a, 300b formed by first and second layers 100, 200, wherein a barrier 310 is utilized in a different manner than shown in FIG. 6. Here, barrier 310 spans the opening, and contacts side surfaces 30s₁, 30s₂ of adjacent struts 30a, 30b, resulting in a substantially horizontal arrangement barrier 310. First pocket 300a containing first therapeutic material 400a is therefore separated from second pocket 300b containing second therapeutic material 400b by barrier 310. Moreover, first layer 100 may contact the length of side surfaces 30s₁, 30s₂, which in this case results in both pockets 300a, 300b being at least partially formed by first layer 100.

FIG. 80 shows pockets 300a, 300b formed by first 100a, 100b and second layers 200a, 200b, wherein the pockets are separate from each other. FIG. 80 is a variation of the embodiments 300a, 300b shown in FIG. 6.

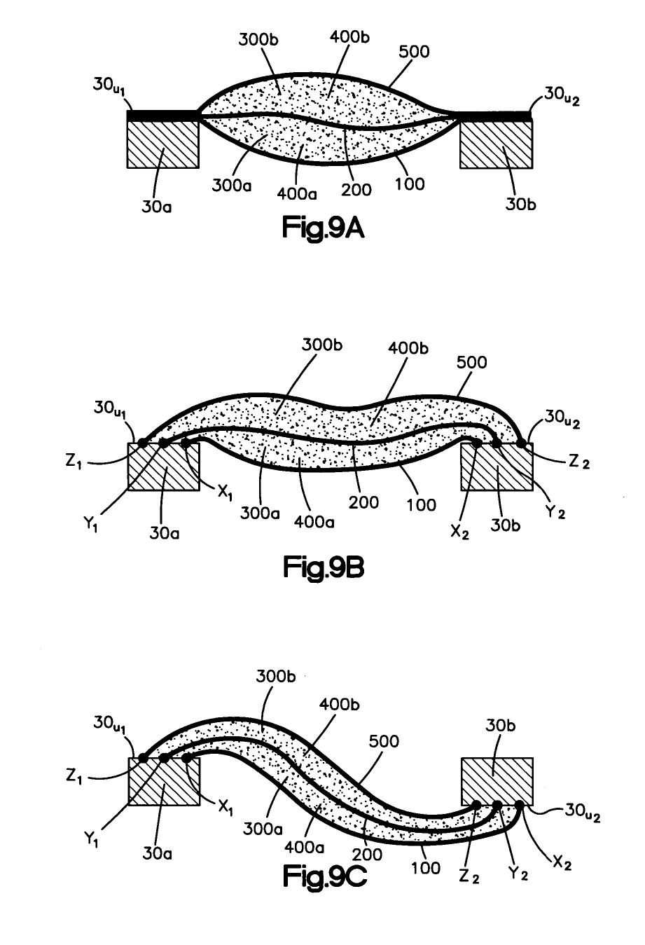

FIGS. 9A-9C are enlarged partial cross-sectional views of pockets 300 formed from first, second, and third layers 100, 200, 500 disposed in openings 40 between adjacent struts 30a, 30b. It is expressly contemplated that a pocket 300 could exhibit some or all of the characteristics of pockets described herein, and in detail below.

FIG. 9A shows pockets 300a, 300b formed by layers 100, 200, 500, wherein each layer is at or near the outer surfaces 30u₁, 30u₂ of adjacent struts 30a, 30b, and each pocket is formed as a bulge between the struts. Pockets 300a, 300b may have different therapeutic materials 400a, 400b, within them, respectively. Second layer 200 may serve as a barrier in this embodiment.

FIG. 9B is similar to the embodiment of FIG. 8J, but includes a third layer 500 to form pocket 300b. Third layer may be bonded to outer surfaces 30u₁, 30u₂ of adjacent struts 30a, 30b at points z₁, z₂, respectively. Pocket 300b may contain a second therapeutic agent 400b.

Similarly, FIG. 9C is similar to the embodiment of FIG. 8L, but includes a third layer 500 to form pocket 300b. Third layer may be bonded to outer and inner surfaces 30u₁, 30i₂ of adjacent struts 30a, 30b at points z₁, z₂, respectively. Pocket 300b may contain a second therapeutic agent 400b.

D. Pockets with Layers Disposed Over Different Sidewall Surfaces

In another embodiment of the invention, a first layer 100 may be disposed over at least a portion of the inner sidewall surface 50, and at least a portion of the first layer 100 may extend over a part of an opening 40. Moreover, a second layer 200 may also be disposed over at least a part of the outer sidewall surface 60. At least a portion of the second layer 200 may be disposed over the portion of an opening 40 that the first layer 100 that extends over. The first layer 100 may define a first surface of a pocket 300 and the second layer may define a second surface of the pocket 300. The pocket 300 may be disposed about at least a portion of the opening 40. A therapeutic agent 400 can be contained in the pocket 300. At least one of the first and second layers 100, 200 may be bound to a sidewall surface 50, 60, which may occur using heat, adhesive materials and/or chemicals, or other methods and materials known by those of skill in the art.

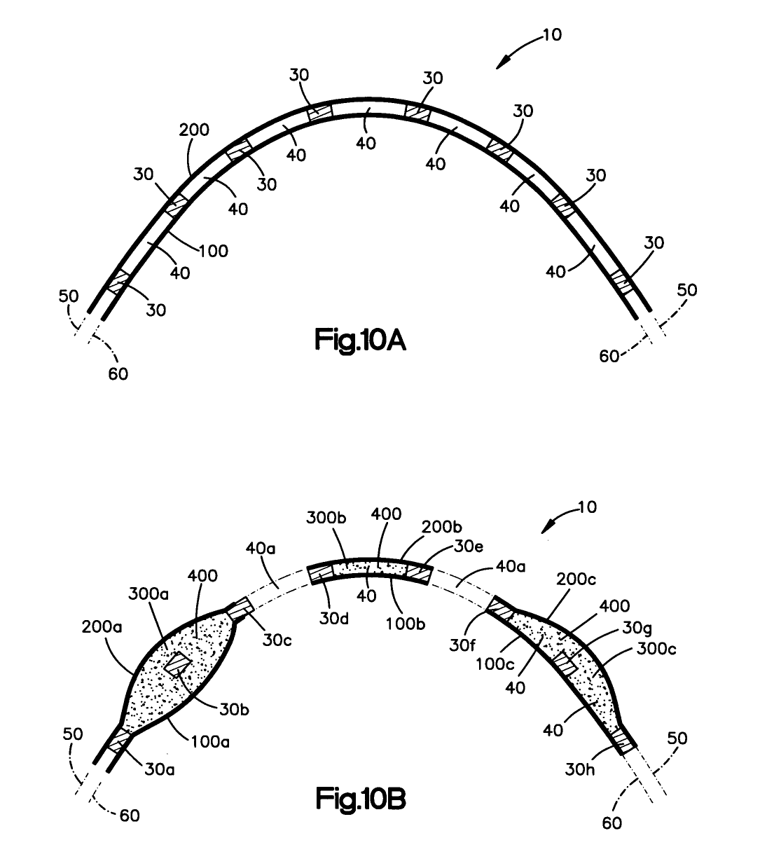

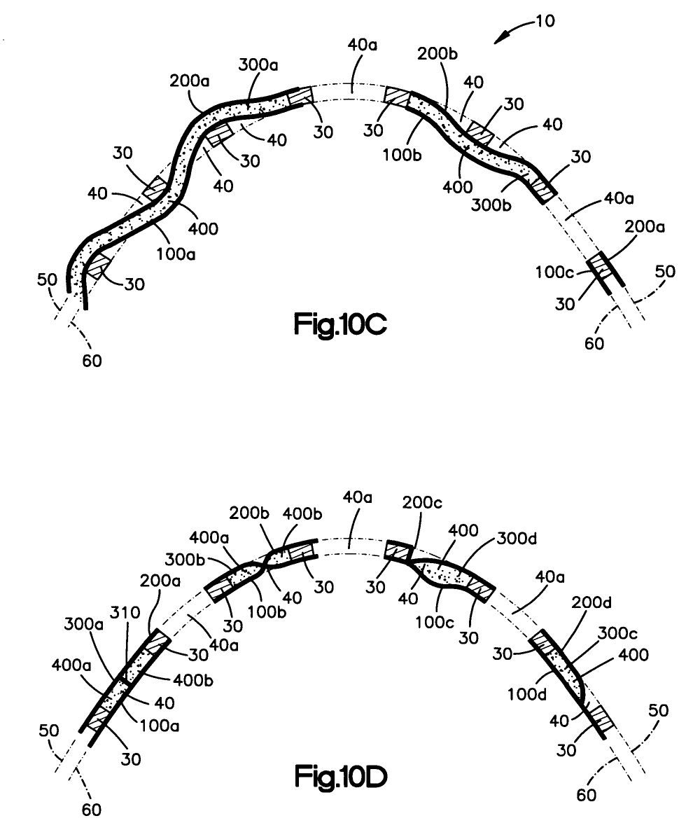

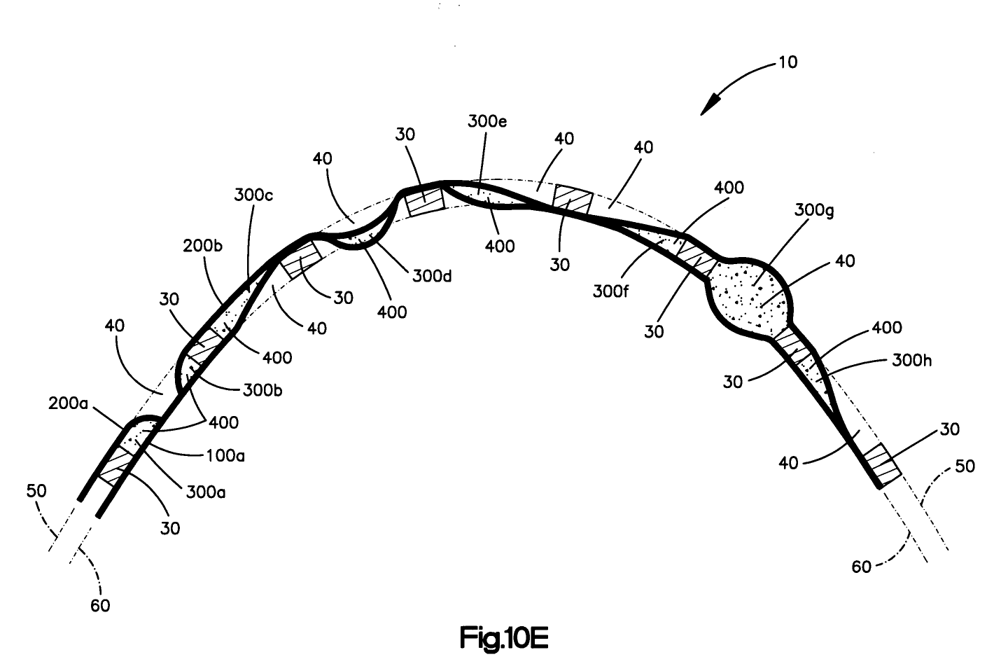

FIG. 10A shows a cross-section of a stent 10 with pockets 300 with first layer 100 disposed on the inner sidewall surface 60 and second layer 200 disposed on the outer sidewall surface 50. The embodiment of FIG. 10A, along with the related embodiments described below in relation to FIGS. 10B-10F, may have any or all of the characteristics described, supra, in relation to the embodiments of FIGS. 1A-9C.

FIG. 10B shows a stent 10 with several examples of pockets 300a, 300b, 300c formed by different sets of first and second layers. Pocket 300a is formed by layers 100a, 200a, and encompasses strut 30b, which is situated between struts 30a and 30c. This embodiment is similar to pocket 300b of FIG. 7. Pocket 300c is also related, except that first layer 100 is in contact with intermediate strut 30g.

FIG. 10C shows further embodiments of pockets 300a, 300b formed by different sets of first and second layers. Pocket 300a results from a “weaved” arrangement of layers 100a, 200a, wherein the first and second layers may contact only every other strut while providing a continuous pocket 300a that is situated about more than one opening 40 and on the outer and inner sidewall surfaces 50, 60. Pocket 300 is a related arrangement, except that the first layer 100b sits at or beneath the inner sidewall surface 60.

FIG. 10D shows further embodiments of pockets 300a-300d formed by different sets of first and second layers 100, 200, which are variations of pockets described supra, but wherein the first 100 and second layers 200 are disposed over different sidewall surfaces. For example, pocket 300a is formed by layers 100a, 200b, with barrier 310 extending therebetween, and two therapeutic materials 400a, 400b disposed therein. Pockets 300b, 300c in this embodiment are formed in a single opening 40, wherein a portion of layers 100b, 200b are bound to each other. Pocket 300d in this embodiment is formed by layers 100c, 200c, wherein a portion of layer 200c is disposed over an entire side surface of an adjacent strut 30. Pocket 300e in this embodiment is formed by layers 100e, 200e, wherein layer 200e does not extend over the entire opening 40.

FIG. 10E is an exemplary stent demonstrating the variations of pockets 300 that may be situated on a single stent 10, using the teachings described above. As seen in the drawing, layers 100, 200 may or may not be continuous, and may or may not extend completely over openings. Moreover, layers 100, 200 may or may not be disposed over the same sidewall surface. Pockets 300 may vary in size and shape from opening to opening.

FIG. 10F shows another embodiment wherein the first and second layers 100, 200 are bound to each other. Each layer 100, 200 can, but need not be bound to a strut 30. For example, layers 100a, 200a are bound to each other at points A₁, A₂, and A₃ to form pockets 300a, 300b. Layers 100a, 200a are not bound to a strut 30, however. Likewise layers 100b, 200b are bound to each other at points B₁ and B₂ to form pocket 300c, which encompasses two struts 30. Layers 100c, 200c are bound to each other at points C₁ and C₂, with layer 200c also bound to a strut 30. Layers 100c, 200c form pocket 300d.

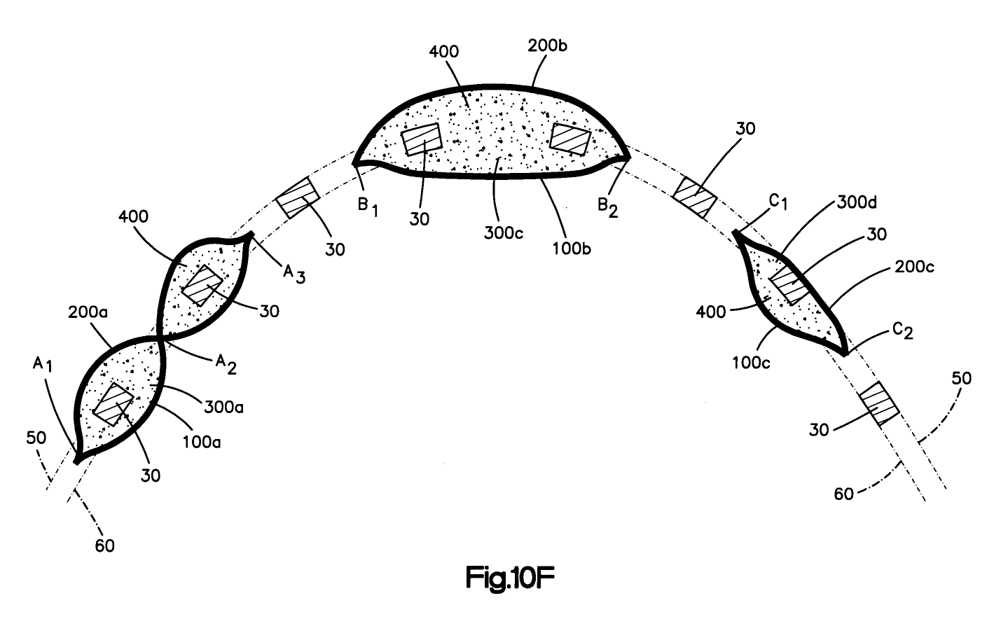

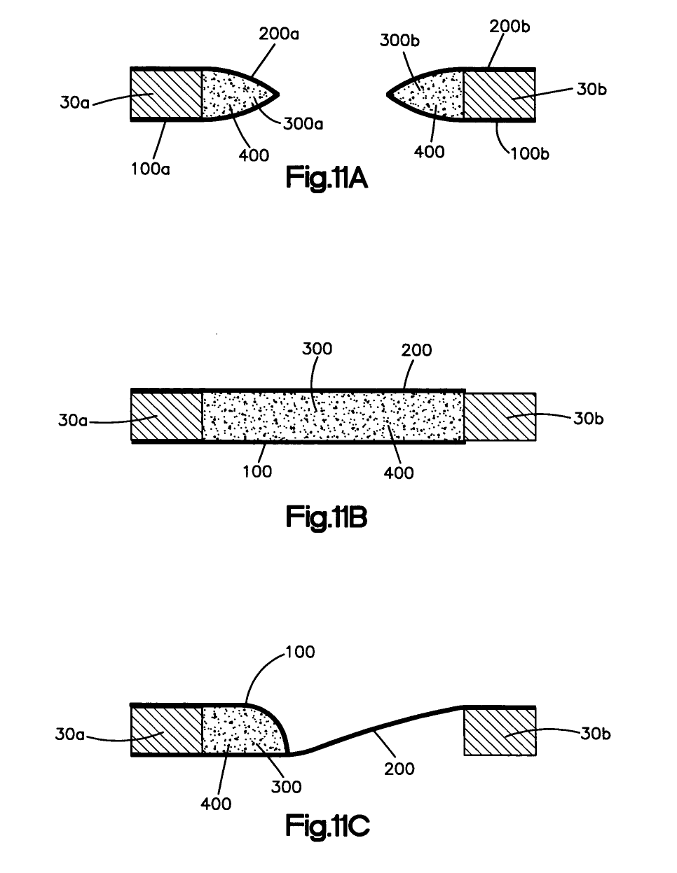

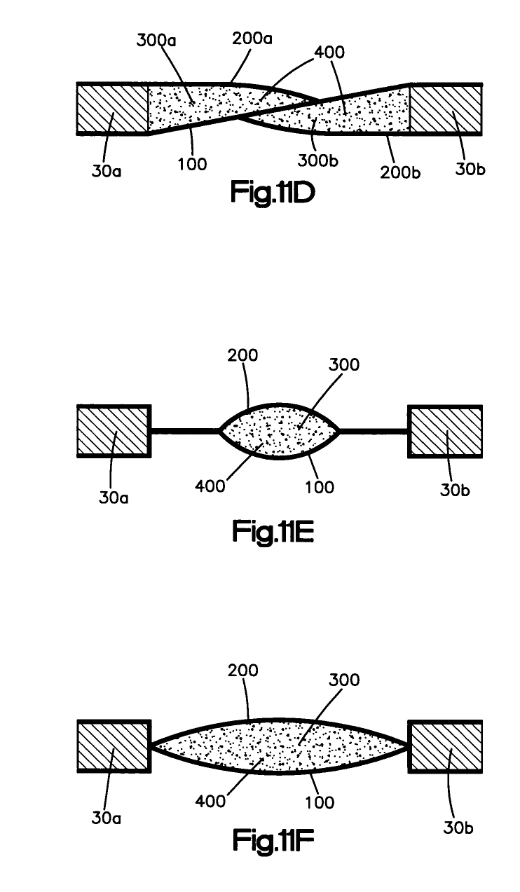

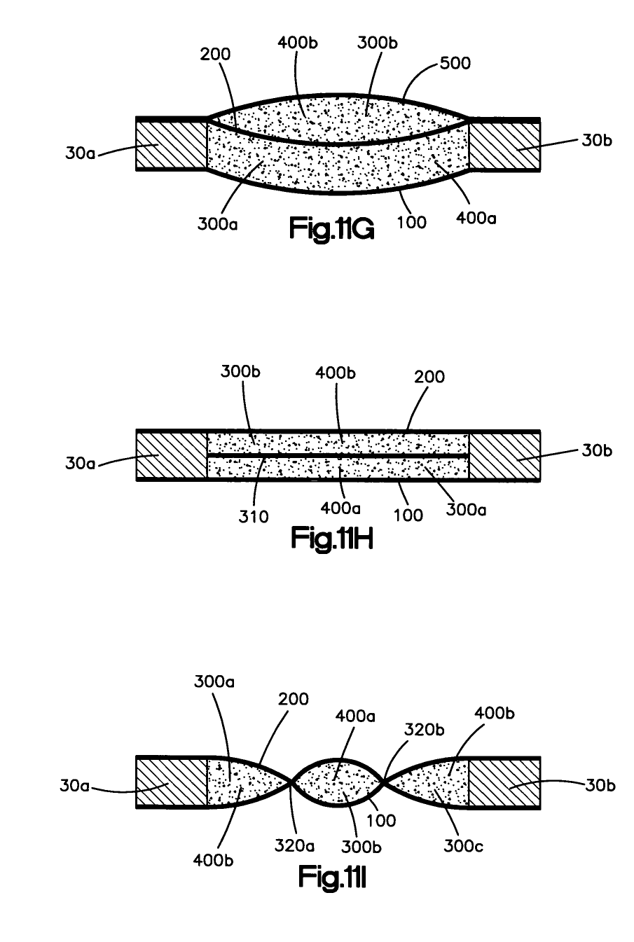

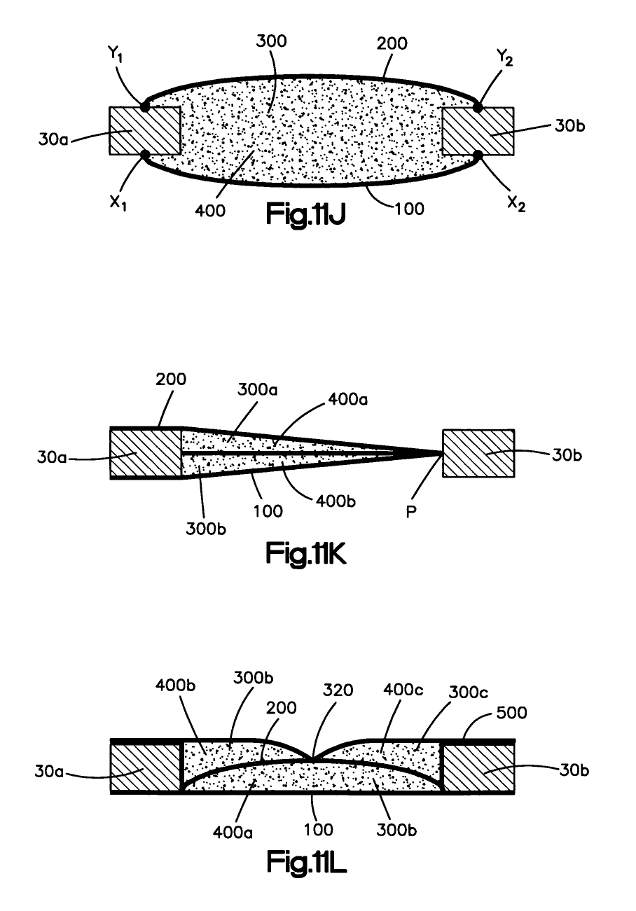

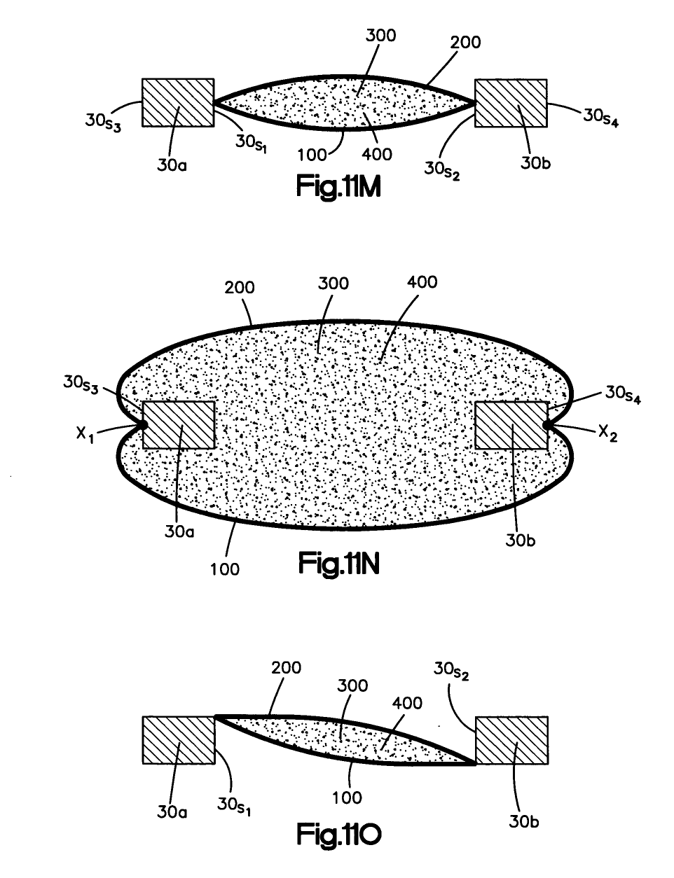

FIGS. 11A-11L are enlarged partial cross-sectional views of pockets 300 formed from first and second layers 100, 200 disposed in openings 40 between adjacent struts 30a, 30b. A first layer 100 may be disposed over a first sidewall and a second layer 200 may be disposed over a second sidewall. It is expressly contemplated that a pocket 300 could exhibit some or all of the characteristics of pockets described herein, and in detail below.

FIG. 11A shows pockets 300a, 300b formed by first 100a, 100b and second layers 200a, 200b, wherein the pockets are separate from each other, and is similar to the arrangement shown in FIG. 8O.

FIG. 11B shows a pocket 300 formed by layers 100, 200, wherein the layers contact opposite sides of adjacent strut 30a, and terminate at the corners of strut 30b. FIG. 11C shows an inverted variation similar to the embodiment of FIG. 8F.

FIG. 11D shows pockets 300a, 300b formed by running a first layer 100 through the opening 40, and disposing second layers 200a, 200b to form the pockets. For this particular embodiment, in addition to others, it may be beneficial to provide a first layer 100 that is relatively thicker or more resilient as compared to second layers 200a, 200b. The characteristics of the layers are discussed in more detail, infra.

FIG. 11E is a variation of the embodiments shown in FIGS. 8B and 8H. FIG. 11F is similar to the embodiment of FIG. 11E, wherein the pocket 300 is enlarged so that the sides of the pocket touch the struts 30a, 30b.

FIG. 11G shows two “stacked” pockets 300a, 300b formed by layers 100, 200, 500. In this embodiment, first layer 100 is disposed along the inner sidewall surface, and second and third layers 200, 500 are disposed along the outer sidewall surface.

FIG. 11H shows an embodiment similar to the arrangements shown in FIG. 10A, wherein pockets 300a, 300b are separated by barrier 310. This embodiment is similar to the one shown in FIG. 8N.

FIG. 11I shows pockets in a “bowtie” arrangement, wherein layers 100, 200 are conjoined at nodes 320a, 320b to form three pockets 300a, 300b, 300c within the opening. In this embodiment, pocket 300b has a first therapeutic material 400a, and pockets 300a and 300c have a second therapeutic material 400b. Nodes 320a, 320b may be rupturable upon expansion of stent 10, or may remain intact as first and/or second layers 100, 200 rupture. Nodes 320a, 320b may also be ruptured by the other methods and materials described herein.

FIG. 11J is a variation of FIG. 8J, but wherein first and second layers 100, 200 are bonded to opposite sides of adjacent struts 30a, 30b.

FIG. 11K is an embodiment of pockets 300a, 300b separated by barrier 310 and formed by convergent layers 100, 200. In this arrangement, layers 100, 200 and barrier 310 all terminate at common point P along side surface 30s₂ of strut 30b.

FIG. 11L is an embodiment of an opening having three pockets 300a, 300b, 300c, containing different therapeutic materials 400a, 400b, 400c, respectively, and centrally conjoined at node 320, which may have any or all of the characteristics described herein.

E. Pockets Formed by Layers Bound to Side Surfaces of Struts

In another embodiment of the invention, pockets 300 are defined by first and second layers 100, 200 that are bound to the side surfaces of struts 30 that make-up the stent sidewall 50, 60. In some embodiments, the layers are bound to the opposing side surfaces 30s₁, 30s₂ of opposing struts 30a, 30b, such as in FIG. 11M. In this embodiment, first layer 100 and second layer 200 are each bound to side surfaces 30s₁, 30s₂ of struts 30a, 30b, respectively. The layers 100, 200 define pocket 300 which contains a therapeutic agent 400.

Alternatively, as shown in FIG. 11N, the layers 100, 200 do not have to be bound to opposing side surfaces of struts 30, but instead can be bound to other side surfaces 30s₃, 30s₄ of struts 30a, 30b at points x₁, x₂. Similarly, the layers 100, 200 can be bound to different combinations of side surfaces.

FIGS. 11O and 11P shows variations of the embodiment of FIG. 11M, wherein the layers 100, 200 are bound to different positions on the side surfaces 30s₁, 30s₂ of struts 30a, 30b. In FIG. 110, the layers 100, 200 are bound near the top of side surface 30s₁, but near the bottom of side surface 30s₂. In FIG. 11P, first layer 100 is bound near the top of side surface 30s₁, and near the bottom of side surface 30s₂. In this embodiment, second layer 200 is bound near the top of side surfaces 30s₁, 30s₂. As shown in these depictions, layers 100, 200 can be bound to various positions along the side surfaces of struts 30.

FIG. 11Q shows an embodiment where layers 100, 200 are not bound to adjacent struts. The pocket 300 formed in this embodiment extends beyond one opening.

FIG. 11R shows an embodiment where layers 100, 200 define two pockets 300a, 300b within a single opening.

F. Exemplary Methods of Use and Making the Invention

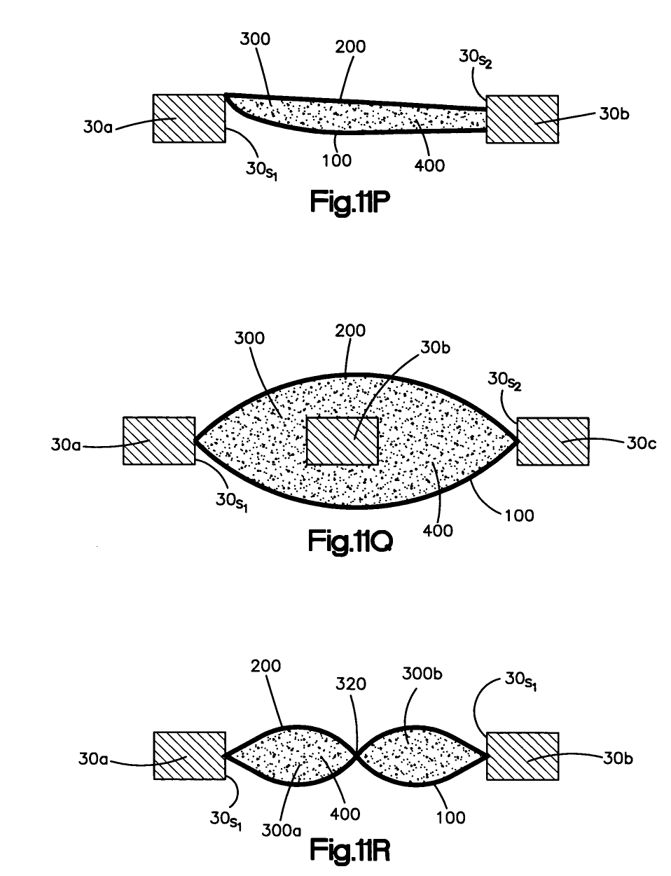

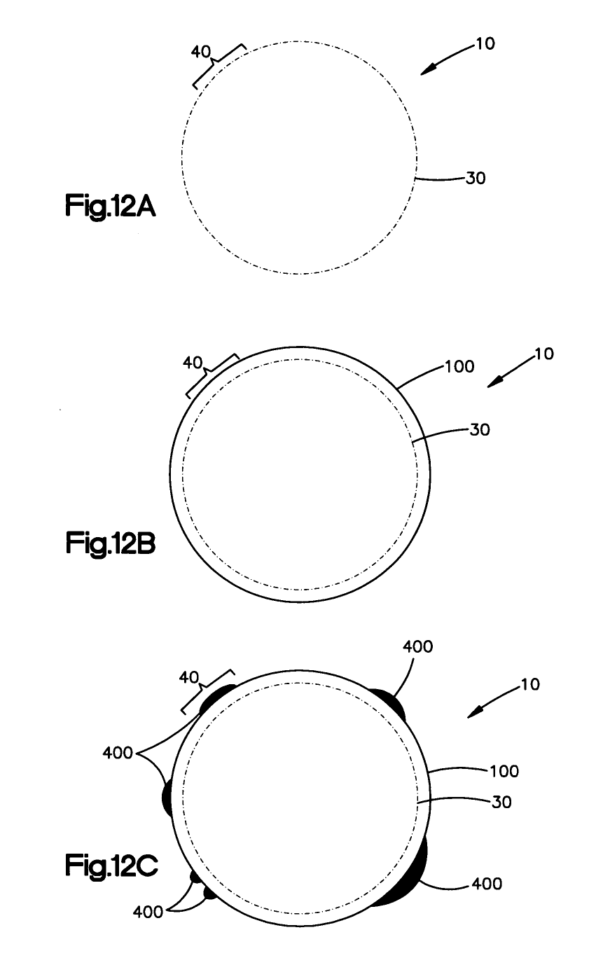

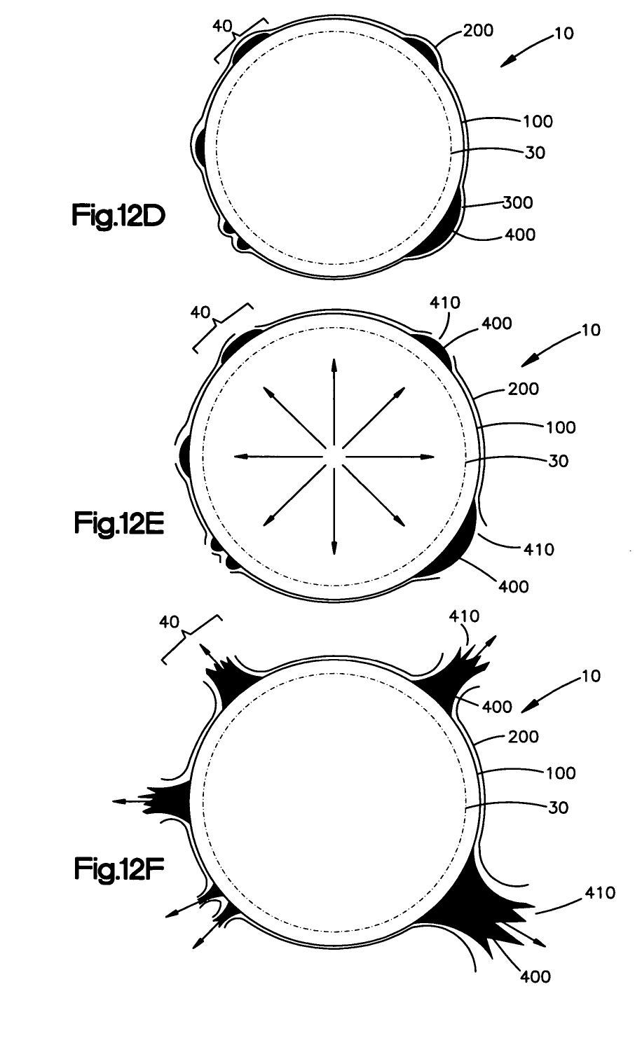

FIGS. 12A-12F illustrate an exemplary process and use of an embodiment of a stent 10 with struts 30 and amounts of therapeutic material 400 which may be disposed within pockets 300. In this embodiment, pockets 300 are formed by layers 100, 200 on the outer sidewall surface 60 (not shown). This method can also be used to form pockets having two layers disposed on the inner sidewall surface 50. FIG. 12A shows a cross-sectional view of a stent 10 with struts 30. Stent 10 has openings 40 between struts 30 (see, e.g., FIG. 1B), and is in a compressed condition. FIG. 12B shows the stent 10 of FIG. 12A after an semi-flexible first layer 100 has been applied. The layers 100, 200 may be bonded to the struts 30 by using techniques known in the art, e.g., adhesives, heat bonding, and ultrasonic welding. The distance between the first layer 100 and the struts 30 is exaggerated to show detail and contrast.

FIG. 12C shows the stent of FIG. 12B after numerous amounts of therapeutic material 400 have been applied to the stent 10 in openings 40. As seen in FIG. 12C, several sizes and shapes of amounts of therapeutic material 400 are utilized with the stent 10. FIG. 12D shows the stent of FIG. 12C after a flexible second layer 200 has been applied, forming pockets 300. The second layer 200 may be bonded to struts 30 and/or the first layer 100. As seen in FIG. 12D, second layer 200 substantially conforms to at least some of the amounts of therapeutic material 400 within pockets 300. The distance between the second layer 200 and the amounts of therapeutic material 400, struts 30, and first layer 100 has been exaggerated to show detail and contrast. FIG. 12E shows the stent of. FIG. 12D in its expanded state. Struts 30, first layer 100, and second layer 200 have all expanded. Importantly, the second layer 200 has also ruptured, shown by ruptures 410, at or near the locations of the amounts of therapeutic material 400 within pockets 300. FIG. 12F shows the stent of FIG. 12E with the content of the amounts of therapeutic material 400 at least partially dispersing away from the stent 10 through ruptures 410 and toward a target site.

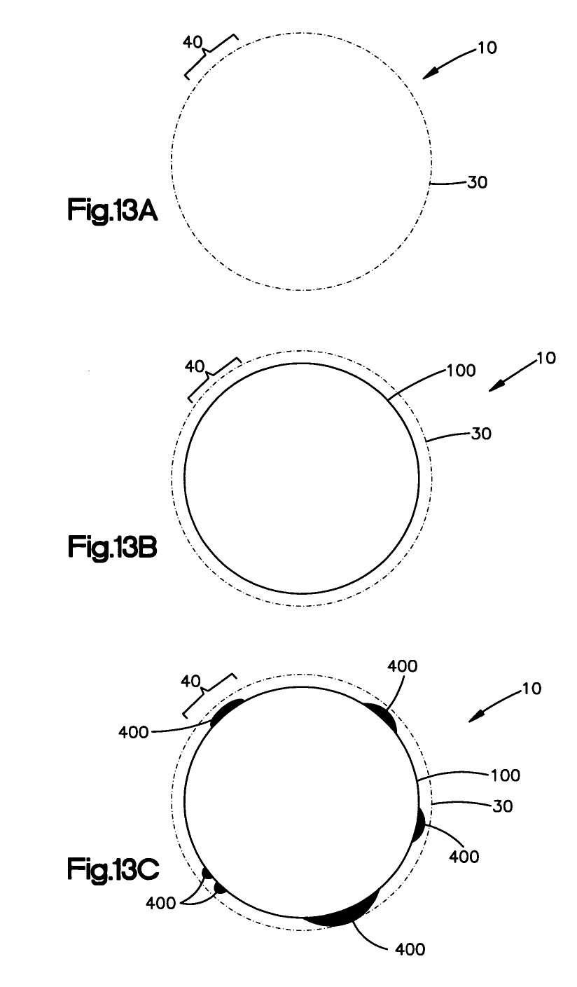

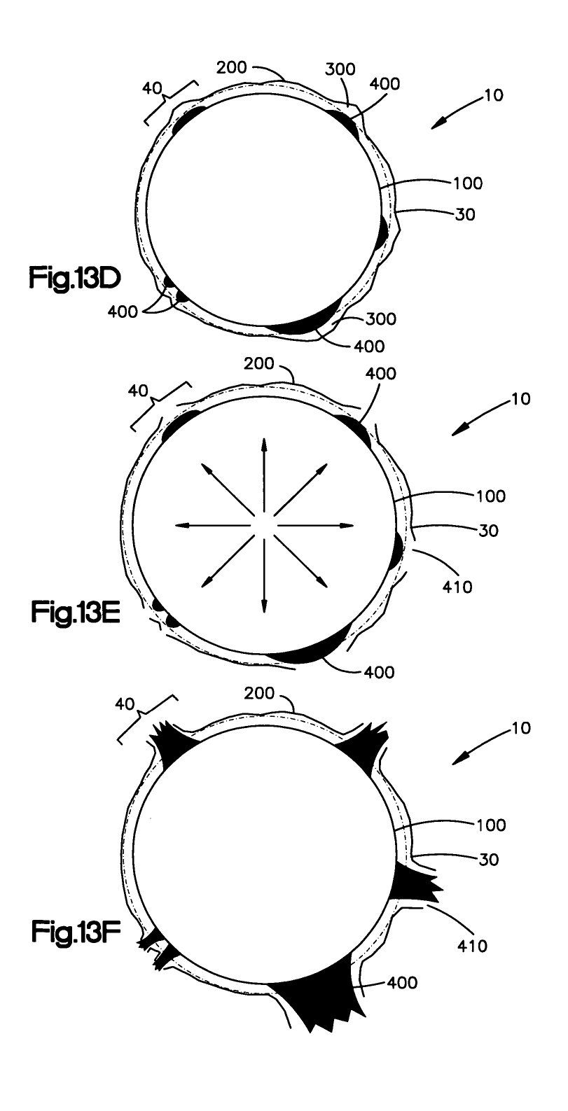

FIGS. 13A-13F show the exemplary method of FIGS. 12A-12F, except that in this embodiment, layers 100, 200 are disposed on different sidewall surfaces 50, 60 (not shown). The description of FIGS. 12A-12F applies to these figures. Also, in one embodiment, the first and/or second layers 100, 200 can be bonded to struts 30. In addition, in some embodiments, the first and second layers 100, 200 are bonded to each other. Again, relative distances between objects may be exaggerated to show detail. In addition to the embodiments of method of use shown in detail, it is expressly contemplated that stent 10 may be applied with layers 100, 200 to form pockets 300 with therapeutic material 400 therein, and subsequently expanded or in some other way manipulated to release therapeutic material 400 from pockets 300. Moreover, such a method may correspond to the numerous embodiments of pockets disclosed herein.

In the embodiment where the first and second layers 100, 200 are bound to the side surfaces of struts 30, the pockets 300 in this embodiment may be formed by affixing the layers 100, 200 to the side surfaces using techniques known in the art. Specifically, a first layer 100 may be affixed to side surfaces of adjacent struts 30, and then a second layer may be affixed to the surfaces of adjacent struts 30, forming at least one pocket 300. The layers 100, 200 can be made of materials used to make the layers 100, 200 of the other embodiments described herein.

Layers 100, 200 may also be applied to a stent 10 in the form of a polymer slurry, which after application to at least a portion of the stent 10, may be allowed to dry and/or cured and form a layer 100, 200 on the stent 10. Layer 100, 200 thickness may be varied by altering the polymer slurry consistency, dip rate, and or curing conditions. A slurry may be applied to the stent 10 in the expanded or unexpanded state.

G. Further Embodiments of Struts

FIGS. 14A-14D are enlarged partial cross-sectional views of pockets 300 formed from first and second layers 100, 200 disposed in openings 40 between adjacent struts 30a, 30b. It is expressly contemplated that a pocket 300 could exhibit some or all of the characteristics of pockets described herein, and in detail below. More specifically, FIGS. 14A-14D are exemplary strut shapes and sizes for use with the present invention.

FIG. 14A shows an embodiment where struts 30a, 30b have a rounded cross-sectional shape. Such a shape may be circular, elliptical, oval, or some combination thereof. FIG. 14B shows an embodiment wherein a rectangular strut 30a is paired with a rounded strut 30b. FIG. 14C shows another embodiment of struts, wherein the struts 30a, 30b are hexagonal. Further suitable cross-sectional strut shapes include squares, parallelograms, triangles, octagons, irregular shapes, or any other polygonal shape. It is further noted that any combination of suitable shapes may be used on a single stent 10.

FIG. 14D shows an embodiment wherein adjacent struts 30a, 30b are of substantially different size. Such size variation may be used with any combination of shapes discussed herein, or that may be appreciated by those skilled in the art.

Overall, it is expressly contemplated that the pocket and layering designs shown and described in reference to the figures herein may be varied and/or combined by those skilled in the art. The designs shown are exemplary and the concepts and variations shown are intended to be viewed as several of the many embodiments contemplated by providing struts and layers to form pockets 300.

H. The Layers

Layers 100, 200 may be composed of one or more sub-layers (not shown). Layers 100, 200 may be comprised of a variety of suitable materials, such as Polyurethane or Silicone, or a suitable polymer. More than one material may be used for individual sub-layers to create a first or second layer 100, 200. First and second layer 100, 200 may be comprised of different materials. Having the first and second layers 100, 200 different materials may also a user to vary the porosity, tear strength, breakdown rate, and/or texture of each layer individually. The selection and variance of these attributes may be beneficial if, for example, it is desirable that the contents of a pocket 300 (such as an amount of therapeutic material 400) are to be delivered through the second layer 200, but preferably not the first layer 100. It may also be desirable to alter the release rates of the contents of a pocket 300 based upon the choice and/or combination of materials and methods used in applying each layer 100, 200 to a stent 10. For instance, the chosen material for a layer may be relatively porous, to allow the contents of the pocket 300 to disperse slowly. A detailed discussion of suitable materials for layers 100, 200 appears below.

The chosen material for each layer may be applied to the stent 10 while in the form of a slurry. Layers 100, 200 may be directly applied to a stent 10 by dispensing a slurry to the stent, or by affixing the stent onto a cylindrical mandrel and dipping the assembly into a slurry. The thickness of each sub-layer or layer may be altered based on the consistency of the slurry, the dipping rate, and/or the curing conditions. Other methods and materials for applying layers to the stent may be utilized as deemed appropriate by one skilled in the art.

Layers 100, 200 may be applied while the stent 10 is in its collapsed or expanded state. If the layers are applied to the stent 10 while the stent is in its collapsed state, the layers should be comprised at least in part of a flexible material that is able to stretch when the stent 10 expands. A layer that is inflexible may undesirably rupture upon the expansion of the stent 10.

Varying flexibility of a layer may also allow for increased or decreased capabilities in pocket volume and dimensions. For example, if the first layer 100 is made of a more rigid material, and the second layer 200 is made of a more flexible material, the pocket may tend to “bulge” outwards, utilizing the increased flexibility of the second layer 200. Such an arrangement may be preferable when it is desirable to maintain the first shape of the stent 10 to, for example, maintain a maximum flow path therethrough. Moreover, it may be preferable to create or supplement a pocket 300 after the layer is complete, by such means as a injecting element. Having at least one layer 100, 200 made of a flexible material may allow a increased amount of content to be inserted into a pocket, as the pocket could “stretch” to increase its volume as its is filled.

Furthermore, it may also be preferable to have increased flexibility with the first layer 100 to pattern the rupture and/or dispersion of the content of the pockets 300 to the inside of the stent 10. This may be assisted by the expansion of a balloon (not shown) inside the stent, the pressure of which against the first layer 100 could cause ruptures and allow for dispersion of the content of the pockets 300 along the inside of the stent 10.

The layers 100, 200 may also be made of a biodegradable material. Similarly, it may be preferable for first and second layer to have varying degrees of biodegradability to assist in controlling the release rate of the content of a pocket.

The layers 100, 200 may also be applied to the stent with pre-cut tears in the layer. The first and/or second layers may have such tears. When the stent expands, as seen in FIGS. 12E, 13E, the tears may localize the points at which a layer ruptures. Therefore, a user may exert increased control over the dispersion pattern and area of the content of the pockets 300 by preselecting the tear and rupture points of layer. Such a design may be especially desirable when the target tissue site for content delivery is very localized, or it is undesirable to deliver the content to areas other than the target site, such as the bloodstream.

As an alternative to making pre-cut tears in a layer to dictate rupture points, a layer may be imprinted by used of a contour mandrel during the layering process. The surface of such a contoured mandrel may not create punctures or tears in the layer upon formation, but instead would imprint patterns of thinner or weaker areas in the layer. Upon expansion, the imprinted areas would preferably be the first areas to rupture.

When using a first layer 100 that is not entirely rigid, it may also be desirable to expand the pockets 300 towards the longitudinal axis of a hollow cylindrical stent. This may be accomplished by simply providing a flexible first layer. The expansion of the pockets 300 toward the longitudinal axis may also be urged by using a hollow cylindrical mandrel, having a longitudinal axis substantially coaxial to the axis, to apply the first layer and subsequently running a vacuum through the mandrel to exert an axial force on the pockets 300, pulling them toward the longitudinal axis.

To assist or cause the rupture of the first and/or second layers, it may also be preferable to place a spike (or other equivalent sharpened element) within a void or pocket to puncture the first and/or second layers when the stent 100 is expanded. The spike may be bioresorbable, and/or may also be part of the strut 30 structure itself. A related embodiment is to provide spikes (or other equivalent sharpened elements) on the balloon itself. When the balloon expands, the spikes on the balloon would then puncture the first and/or second layers, allowing the content of the affected pockets 300 to disperse. Such balloons are known in the art as infiltrating balloons or cutting balloons. When using such a balloon, it may be preferable to make the first layer 100 and/or second layer 200 of a self-sealing material, to enable the first layer to close its punctures after the balloon has retracted.

As an alternative to using a balloon or other expanding device to rupture pockets 300 of a stent 10, pockets may be ruptured locally by the use of an ultrasonic device. In such an embodiment, the pockets 300 could have therapeutically-loaded microbubbles which would burst in response to an ultrasonic impetus.

Stent 10 may also be ruptured by way of a time-delayed decay. In such an embodiment, at least one layer would be at least partially comprised of a biodegradable material, which would be configured to decay over a predetermined period of time to eventually release a therapeutic agent.

More than one stent 10 may also be arranged in a combination or matrix format. Such uses of more than one stent 10 are known in the art.

It should be noted as well that the use of layers with a stent may also be beneficial in protecting the contents of the pocket, the stent itself, and any expansive device (such as a balloon) during the implantation of the assembly into the body. Stents directly coated with therapeutic agents can lose significant quantities of their agent during implantation, as the stent will often come into contact with vessel walls, bodily fluids, etc. before reaching the target site. The use of layers over the pockets may help guard against such a loss of therapeutic material.

I. Therapeutic Agents

The contents of a pocket 300 and/or coating may contain one or more biological active materials, such as an amount of therapeutic material 400. The term “biologically active material” encompasses therapeutic agents, such as biologically active agents, and also genetic materials and biological materials. The term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of bNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

The solvent that is used to form the coating composition include ones which can dissolve the polymer into solution and do not alter or adversely impact the therapeutic properties of the therapeutic agent employed. Examples of useful solvents include tetrahydrofuran (THF), methyl ethyl ketone chloroform, toluene, acetone, issoctane, 1,1,1-trichloroethane, isoppropanol, IPA and dichloromethane or mixtures thereof.

J. Coating the Stent

It may be beneficial to apply a coating to a stent 10 with pockets 300. The coating can be applied over the layers 100, 200 forming pockets 300, and/or over parts of the stent 10 that are not covered by a layer 100, 200. A coating composition may be prepared, for example, by applying a mixture of a polymeric material, a solvent and a therapeutic agent on a surface to form a coating. If such a composition is used the polymeric material incorporates the therapeutic agent. Alternatively, the coating composition may not include a polymeric material. The following is a description of suitable materials and methods useful in producing a coating on the surface of stent struts of the invention.

Polymeric materials useful for forming the coating should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers. Since the polymer is being applied to a part of the medical device which undergoes mechanical challenges, e.g. expansion and contraction, the polymers are preferably selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. The polymer is selected to allow the coating to better adhere to the surface of the strut when the stent is subjected to forces or stress. Furthermore, although the coating can be formed by using a single type of polymer, various combinations of polymers can be employed.

Generally, when a biologically active material used is a hydrophilic, e.g., heparin, then a matrix material comprising a more hydrophilic material has a greater affinity for the biologically active material than another matrix material that is less hydrophilic. When a biologically active material used is a hydrophobic, e.g., paclitaxel, actinomycin, sirolimus (RAPAMYCIN), tacrolimus, everolimus, and dexamethasone, then a matrix material that is more hydrophobic has a greater affinity for the biologically active material than another matrix material that is less hydrophobic.

Examples of suitable hydrophobic polymers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by RₐSiO_{4-a/2}, where R is a monovalent substituted or unsubstituted hydrocarbon radical and the value of a is 1 or 2; and naturally occurring hydrophobic polymers such as rubber.

Examples of suitable hydrophilic monomer include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl(meth)acrylate and 2-hydroxypropyl(meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Suitable stents may also be coated or made with non-polymeric materials. Examples of useful non-polymeric materials include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides.

Coating compositions can be applied by any method to a surface of a medical device to form a coating layer. Examples of suitable methods include, but are not limited to, spraying such as by conventional nozzle or ultrasonic nozzle, dipping, rolling, electrostatic deposition, and a batch process such as air suspension, pan coating or ultrasonic mist spraying. Also, more than one coating method can be used to make a medical device. Coating compositions suitable for applying a coating to the devices of the present invention can include a polymeric material dispersed or dissolved in a solvent suitable for the medical device, wherein upon applying the coating composition to the medical device, the solvent is removed. Such systems are commonly known to the skilled artisan.

A coating of a medical device of the present invention may include multiple coating layers. For example, the first layer and the second layer may contain different biologically active materials. Alternatively, the first layer and the second layer may contain an identical biologically active material having different concentrations. In one embodiment, either of the first layer or the second layer may be free of biologically active material. For example, when the biologically active solution is applied onto a surface and dried (the first layer), a coating composition free of a biologically active material (the second layer) can be applied over the dried biologically active material.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein by reference, in their entirety, for all purposes related to this disclosure.

While the invention has been shown and described herein with reference to particular embodiments, it is to be understood that the various additions, substitutions, or modifications of form, structure, arrangement, proportions, materials, and components and otherwise, used in the practice and which are particularly adapted to specific environments and operative requirements, may be made to the described embodiments without departing from the spirit and scope of the present invention. Accordingly, it should be understood that the embodiments disclosed herein are merely illustrative of the principles of the invention. Various other modifications may be made by those skilled in the art which will embody the principles of the invention and fall within the spirit and the scope thereof.

What is claimed is:

1. A medical device for delivering a therapeutic agent comprising:
(a) a stent having a sidewall comprising a plurality of struts, at least a first opening in the sidewall, and a first sidewall surface at least partially defined by the plurality of struts and the first opening;
(b) a first layer disposed over at least a part of the first sidewall surface, wherein at least a portion of the first layer extends over a part of the first opening;
(c) a second layer disposed over at least a part of the first sidewall surface, wherein at least a portion of the second layer is disposed over the portion of the first layer that extends over the first opening,
(d) at least a first pocket disposed about at least a portion of the first opening; wherein the pocket is defined at least in part by the first layer and at least in part by the second layer; and
(e) a therapeutic agent contained in the first pocket.

2. The medical device of claim 1, wherein the first layer is bound to the stent.

3. The medical device of claim 1, wherein the second layer is bound to the first layer.

4. The medical device of claim 1, wherein the first pocket is co-extensive with the first opening.

5. The medical device of claim 1, wherein the first pocket is within the first opening.

6. The medical device of claim 1, wherein the first pocket extends beyond the first opening.

7. The medical device of claim 1, further comprising a second pocket disposed about the first opening.

8. The medical device of claim 1, wherein at least one strut comprises a side surface and the first layer is disposed over the side surface.

9. The medical device of claim 1, wherein at least one strut comprises a side surface and the second layer is disposed over the side surface.

10. The medical device of claim 1, wherein at least one strut comprises a side surface and the first and second layers are disposed over the side surface.

11. The medical device of claim 1, wherein the sidewall further comprises a second sidewall surface and a third layer is disposed over the second sidewall surface.

12. The medical device of claim 11, wherein the sidewall further comprises a fourth layer disposed over at least a portion of the third layer.

13. The medical device of claim 1, wherein the stent further comprises a second opening, and wherein a second pocket is disposed about the second opening.

14. The medical device of claim 13, wherein the second pocket is defined at least in part by the first layer and at least in part by the second layer.

15. The medical device of claim 13, wherein the second pocket is defined at least in part by a third layer and at least in part by a fourth layer.

16. The medical device of claim 15, wherein the third layer is disposed over at least a portion of the first sidewall surface, and the fourth layer is disposed over at least a portion of the second sidewall surface.

17. The medical device of claim 13, wherein the second pocket contains a therapeutic material.

18. The medical device of claim 1, further comprising a second pocket, and wherein the second pocket contains a different therapeutic material than the first pocket.

19. The medical device of claim 18, wherein the second pocket is disposed about the first opening.

20. The medical device of claim 18, further comprising a second pocket, and wherein the first and second pockets are interconnected.

21. The medical device of claim 1, wherein at least one of the first and second layers comprises a plurality of sub-layers.

22. The medical device of claim 21, wherein at least two sub-layers are comprised of a different material.

23. The medical device of claim 21, wherein at least two sub-layers are of different thicknesses.

24. The medical device of claim 1, further comprising a barrier between the first and second layers.

25. The medical device of claim 1, further comprising a third layer disposed over at least a portion of one of the first and second layers.

26. The medical device of claim 1, wherein the first and second layers are comprised of the same material.

27. The medical device of claim 1, wherein the first and second layers are comprised of different materials.

28. The medical device of claim 1, wherein the first and second layers have different tensile strengths.

29. The medical device of claim 1, wherein the first and second layers are of different thicknesses.

30. The medical device of claim 1, wherein at least one of the first and second layer is capable of being ruptured by the expansion of the stent.

31. The medical device of claim 1, wherein at least a portion of at least one of the first and second layer comprises a plurality of pores.

32. The medical device of claim 1, wherein at least one of the first layer and second layer comprise at least one preformed imprint, and wherein the imprinted area generally have a lower tensile strength than the remainder of the layer.

33. The medical device of claim 1, wherein at least one of the first layer and second layer comprises a self-sealing material.

34. The medical device of claim 1, wherein at least one of the first layer and second layer comprises a biodegradable material.

35. The medical device of claim 1, wherein at least one of the first layer and second layer are substantially flexible.

36. The medical device of claim 1, wherein the therapeutic agent is releasable from the first pocket through at least one of the first layer and second layer.

37. The medical device of claim 1, wherein the therapeutic agent is releasable from the first pocket after the expansion of the stent.

38. A medical device for delivering a therapeutic agent comprising:
(a) a stent having a sidewall comprising a plurality of struts, at least a first opening in the sidewall, an outer sidewall surface at least partially defined by the plurality of struts and the first opening, and an inner sidewall surface at least partially defined by the plurality of struts and the first opening;
(b) a first layer disposed over at least a portion of the outer sidewall surface, wherein at least a portion of the first layer extends over a part of the first opening, and wherein the first layer is bound to at least a portion of the stent;
(c) a second layer disposed over at least a portion of the inner sidewall surface, wherein at least a portion of the second layer extends over the first opening,
(d) at least a first pocket disposed about at least a portion of the opening; wherein the pocket is defined at least in part by the first layer and at least in part by the second layer; and
(e) a therapeutic agent contained in the first pocket.

39. A medical device for delivering a therapeutic agent comprising:
(a) a stent having a sidewall comprising a plurality of struts, at least a first opening in the sidewall, an outer sidewall surface defined by the plurality of struts and the opening, and an inner sidewall surface defined at least partially by the plurality of struts and the opening;
(b) a first layer disposed over at least a portion of the outer sidewall surface, wherein at least a portion of the first layer extends over a part of the first opening;
(c) a second layer disposed over at least a part of the inner sidewall surface, wherein at least a portion of the second layer extends over the opening, and wherein the second layer is bound to at least a portion of the first layer,
(d) at least a first pocket disposed about at least a portion of the opening; wherein the pocket is defined at least in part by the first layer and at least in part by the second layer; and
(e) a therapeutic agent contained in the first pocket.

40. A medical device for delivering a therapeutic agent comprising:
(a) a stent comprising a sidewall comprising at least a first strut and a second strut, and at least a first opening in the sidewall, wherein the first strut and the second strut each comprise an outer surface, an inner surface and at least one side surface;
(b) a first layer bound to a side surface the first strut and bound to a side surface of the second strut, wherein at least a portion of the first layer extends over a portion of the first opening;
(c) a second layer bound to a side surface of the first strut and bound to a side surface of the second strut, wherein at least a portion of the second layer extends over a portion of the first opening;
(d) at least a first pocket disposed about at least a portion of the opening; wherein the pocket is defined at least in part by the first layer and at least in part by the second layer; and
(e) a therapeutic agent contained in the first pocket.
